(19) United States
(12) Patent Application Publication
(10) Pub. No.: US 2017/0057948 A1
(43) Pub. Date: Mar. 2, 2017

(54) INDOLE-LIKE TRK RECEPTOR ANTAGONISTS

(71) Applicants: Tallinn University of Technology, Tallinn (EE); University of Tartu, Tartu (EE)

(72) Inventors: Tonis Timmusk, Tallinn (EE); Margus Lopp, Tallinn (EE); Eero Vasar, Tartu (EE); Allen Kaasik, Tartu (EE); Mati Karelson, Tartu (EE)

(21) Appl. No.: 15/249,390

(22) Filed: Aug. 27, 2016

Related U.S. Application Data
(60) Provisional application No. 62/210,661, filed on Aug. 27, 2015.

(51) Int. Cl. C07D 403/06; C12N 15/85; G01N 33/566; C12N 9/12

(52) U.S. Cl. CPC C07D 403/06; C12N 9/12; C12Y 207/10; C12N 15/85; G01N 33/566; G01N 2333/91205; G01N 2333/71; G01N 2333/90241

(57) ABSTRACT

A tropomyosin receptor kinase (Trk) antagonist having a compound of formula (I) or a pharmaceutically acceptable salt thereof,

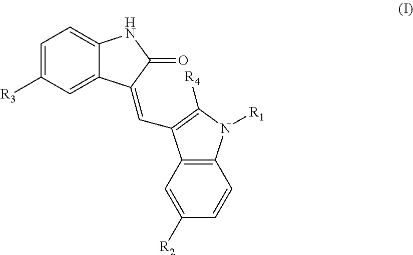

wherein R1 is CH₃, R2 is OCH₃, R3 is SO₂N(CH₃)₂, and R4 is H; or R1 is CH₃, R2 is OH, R3 is SO₂N(CH₃)₂, and R4 is H.

### CROSS REFERENCE TO RELATED APPLICATIONS

This invention claims benefit of priority to U.S. Patent Application No. 62/210,661, filed Aug. 27, 2015; the entire content of which is herein incorporated by reference in its entirety.

### TECHNICAL FIELD

The invention relates to compounds for the modulation of kinase molecules and more specifically to antagonists for inhibiting tropomyosin receptor kinase activity in cells.

### BACKGROUND OF THE INVENTION

The tropomyosin receptor kinase (Trk) family includes three homologous receptor tyrosine kinases: TrkA, TrkB, and TrkC, that specifically bind the neurotrophins nerve growth factor (NGF), brain-derived neurotrophic factor (BDNF), and neurotrophin 4 (NT4) and neurotrophin 3 (NT3), respectively. Activation of Trk receptors by the neurotrophins plays an important role in diverse biological responses, including differentiation, proliferation, survival, and other functional regulation of cells.

Neurotrophins are proteins that modulate the growth, maintenance, and survival of neurons. In addition, NGF and BDNF function as key contributors in chronic neuropathic pain as well as hyperalgesia related to diverse pain states. Hereditary sensory and autonomic neuropathy type V (HSANV) is caused by mutations in NGF gene, leading to the loss of ability to perceive deep pain. Mutations in its receptor TrkA result in HSANIV, which is characterized by congenital insensitivity to pain, anhidrosis, and mental retardation.

Neurotrophin receptors have also been found to play an important role in the development and progression of tumor cells. Alterations in Trk receptor expression, genomic rearrangements or mutations in the gene have been reported in different human cancers, e.g. pancreatic, prostate, breast, ovarian carcinoma, malignant melanomas, thyroid, and neuroblastoma. Interestingly, TrkB receptor has been described to act as a suppressor of anoikis, a type of apoptosis important in prevention of metastasis, highlighting the importance of Trk receptor activity in tumor progression and formulating Trk receptors as potent targets of cancer therapy.

Changes in BDNF and its receptor expression are important in several central nervous system disorders, most notably the enhanced signaling in epilepsy and decreased or increased (depending on the brain region) levels in depression. For this reason, inhibition of TrkB has been proposed as a candidate therapy for epilepsy. In a mouse model, inhibition of TrkB prevented recurrent seizures and alleviated anxiety-like behavior accompanied with a lower level of destructed hippocampal neurons.

Neurotrophins and their receptors have also been implicated to be important in age-related changes in cognition and Alzheimer's disease (AD). Although reports in this field are conflicting with some describing elevated levels of NGF in AD, the prevalent opinion seems to be that increasing the level of NGF or the activity of TrkA is therapeutic for AD. Therefore, all inhibitors of Trk receptors might inflict unwanted side-effects if they are able to cross the blood-brain barrier.

In recent studies, several small molecule TrkA inhibitors have been shown to be effective in neuropathic and inflammatory pain models, able to attenuate cancer-induced pain as well as to block the development of some tumor cells. Therefore, the synthesis or identification of improved Trk-selective inhibitors may provide a therapeutic treatment for Trk related disorders and conditions.

### SUMMARY OF THE INVENTION

The invention addresses the need for compounds and pharmaceutical formulations thereof that function as kinase inhibitors, in particular as antagonists of the tropomyosin receptor kinase (Trk) family. The invention also includes methods of modulating Trk and methods for treating medical conditions mediated at least in part by Trk.

In one aspect of the invention, a composition is provided, which includes formula (I) or a pharmaceutically acceptable salt thereof, where formula (I) is

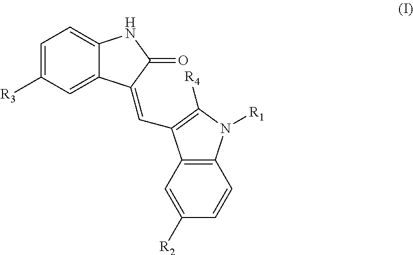

and where in formula (I), R1 is a lower alkyl; R2 is a hydroxy or lower alkoxy; R3 is SO₂N(CH₃)₂; and R4 is H. The lower alkyl is a straight or branched alkyl group having 1 to 6 carbon atoms, preferably 1-3 carbon atoms, more preferably 1 to 2 carbon atoms and most preferably a methyl. The lower alkoxy is a straight or branched alkoxy having 1 to 5 carbon atoms, preferably 1-3 carbon atoms, more preferably 1 carbon atom. In a preferred embodiment R1 is CH₃ and R2 is OCH₃. In another preferred embodiment, R1 is CH₃ and R2 is OH.

In a related aspect of the invention, a composition is provided, which includes formula (II) or a pharmaceutically acceptable salt thereof, where formula (II) is

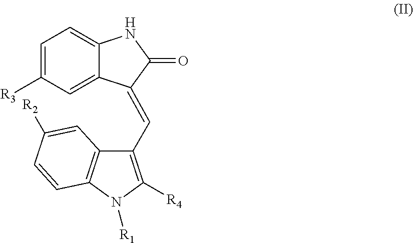

and in formula (II), R1 is a lower alkyl; R2 is a hydroxy or lower alkoxy; R3 is SO₂N(CH₃)₂; and R4 is H. The lower alkyl is a straight or branched alkyl group having 1 to 6 carbon atoms, preferably 1-3 carbon atoms, more preferably 1 to 2 carbon atoms and most preferably a methyl. The lower alkoxy is a straight or branched alkoxy having 1 to 5 carbon atoms, preferably 1-3 carbon atoms, more preferably 1 carbon atom. In some embodiments, R1 is CH₃ and R2 is OCH₃. In other embodiments R1 is CH₃ and R2 is OH.

In a related aspect, the invention provides a method of inhibiting kinase activity of tropomyosin receptor kinase (Trk), which includes providing a cell population expressing a functional Trk, and adding a composition having the formula (I) or a pharmaceutically acceptable salt thereof to the cell population. In some embodiments the method also includes exposing the cell population to an agonist of the Trk. In a preferred embodiment R1 is CH₃; R2 is OCH₃; R3 is SO₂N(CH₃)₂; and R4 is H. In another preferred embodiment, R1 is CH₃; R2 is OH; R3 is SO₂N(CH₃)₂; and R4 is H.

In some embodiments, the composition is added in an amount sufficient to inhibit kinase activity of the Trk, where the Trk is selected from the group consisting of TrkA, TrkB and TrkC, to 18% or less residual activity compared to kinase activity without the composition or compared to kinase activity in the presence of agonist alone. In further embodiments, the composition is added in an amount sufficient to inhibit kinase activity of the Trk, where the Trk is selected from the group consisting of TrkA, TrkB and TrkC, to 10% or less residual activity compared to kinase activity without the composition or compared to kinase activity in the presence of agonist alone.

In another related aspect, the invention includes a method of inhibiting kinase activity of a tropomyosin receptor kinase (Trk), which includes providing a cell population expressing a functional Trk, and adding to the cell population a composition having the Z isomer of formula (I) or the E isomer of formula (II) in an amount sufficient to inhibit kinase activity of the Trk; and optionally exposing the cell population to an agonist of the Trk. In formula (I) and formula (II), R1 is H or a lower alkyl; R2 is selected from the group consisting of H, a hydroxy, and a lower alkoxy; R3 is selected from the group consisting of H, SO₂NH₂ or SO₂NH(CH₃); and R4 is H. In some embodiments, the composition is provided in a pharmaceutically acceptable carrier.

In preferred embodiments the isomer is the Z isomer, R1 is CH₃, R2 is H or OCH₃, and R3 is SO₂NH₂. In further embodiments the composition is added in an amount sufficient to inhibit kinase activity of Trk, where the Trk is selected from the group consisting of TrkA, TrkB and TrkC, to 10% or less residual activity compared to kinase activity without the composition or compared to kinase activity in the presence of agonist alone. In still further embodiments, the composition is added in an amount sufficient to inhibit kinase activity of Trk, where the Trk is selected from the group consisting of TrkA, TrkB and TrkC, to 3% or less residual activity compared to kinase activity without the composition or compared to kinase activity in the presence of agonist alone.

In other preferred embodiments the isomer is the Z isomer, R1 is H, R2 is OCH₃, and R3 is SO₂NH₂. In further embodiments, the composition is added in an amount sufficient to inhibit kinase activity of Trk, where the Trk is selected from the group consisting of TrkA, TrkB and TrkC, to 10% or less residual activity compared to kinase activity without the composition or compared to kinase activity in the presence of agonist alone. In still further embodiments the composition is added in an amount sufficient to inhibit kinase activity of Trk, where the Trk is selected from the group consisting of TrkA, TrkB and TrkC, to 6% or less residual activity compared to kinase activity without the composition or compared to kinase activity in the presence of agonist alone.

In still other preferred embodiments, the isomer is the Z isomer, R1 is CH₃, R2 is OCH₃, and R3 is SO₂NH(CH₃). In further embodiments, the composition is added in an amount sufficient to inhibit kinase activity of Trk to 10% or less residual activity compared to kinase activity without the composition or compared to kinase activity in the presence of agonist alone. In still further embodiments, the composition is added in an amount sufficient to inhibit kinase activity of Trk, where the Trk is selected from the group consisting of TrkA, TrkB and TrkC, to 4% or less residual activity compared to kinase activity without the composition or compared to kinase activity in the presence of agonist alone.

In still another related aspect, the invention provides, a method of inhibiting kinase activity in a cell population, the method including providing a cell population expressing at least one functional kinase selected from the group consisting of Calcium/calmodulin-dependent protein kinase kinase 2 (CaMKK2), Mitogen-activated protein kinase kinase kinase 11 (Map3K11), tropomyosin receptor kinase A (TrkA), tropomyosin receptor kinase B (TrkB), and tropomyosin receptor kinase C (TrkC); and adding to the cell population a composition having a compound of formula (I) in an amount sufficient to inhibit kinase activity; and optionally exposing the cell population to an agonist of the kinase, wherein formula (I) is

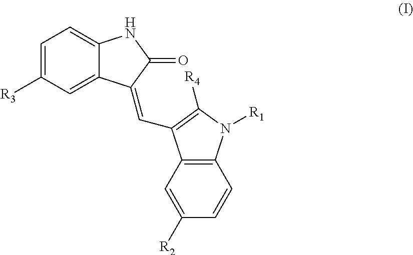

further wherein R1 is CH₃, R2 is OCH₃, R3 is SO₂N(CH₃)₂, and R4 is H.

In still another related aspect, the invention provides, a method of inhibiting kinase activity in a cell population, the method including providing a cell population expressing at least one functional kinase selected from the group consisting of Aurora-B, Calcium/calmodulin-dependent protein kinase kinase 2 (CaMKK2), lymphocyte protein tyrosine kinase (LCK), tropomyosin receptor kinase A (TrkA), tropomyosin receptor kinase B (TrkB), and tropomyosin receptor kinase C (TrkC); and adding to the cell population the composition having a compound of formula (I), in an amount sufficient to inhibit kinase activity; and optionally exposing the cell population to an agonist of the kinase, wherein formula (I) is

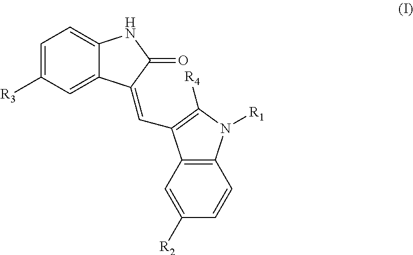

further wherein R1 is CH₃, R2 is OH, R3 is SO₂N(CH₃)₂, and R4 is H.

In still another related aspect, the invention provides, a method of inhibiting kinase activity in a cell population, the method including providing a cell population expressing at least one functional kinase selected from the group consisting of Aurora-B, Calcium/calmodulin-dependent protein kinase kinase 2 (CaMKK2), Check point kinase 2 (Chk2), Erb-B4 receptor tyrosine kinase 4 (Erb-B4), interleukin-1 receptor associated kinase 4 (Irak4), lymphocyte protein tyrosine kinase (LCK), Mitogen-activated protein kinase kinase kinase 11 (Map3K11), Receptor interacting serine/threonine kinase 2 (RIPK2), Serine threonine kinase 1 (Sgk1), SYN aa1-635; and adding to the cell population a composition having a compound of formula (I), optionally in a pharmaceutically acceptable carrier, in an amount sufficient to inhibit kinase activity; and optionally exposing the cell population to an agonist of the kinase, wherein formula (I) is

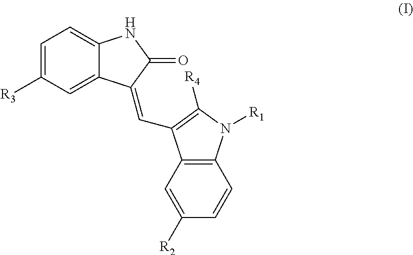

further wherein,
if the kinase is Aurora-B, R1 is CH₃, R2 is H, and R3 is SO₂NH₂, and R4 is H; or R1 is CH₃, R2 is OCH₃, and R3 is SO₂NH₂, and R4 is H; or R1 is H, R2 is OCH₃, R3 is SO₂NH₂, and R4 is H;
if the kinase is CAMKK2, R1 is CH₃, R2 is H, and R3 is SO₂NH₂, and R4 is H; or R1 is CH₃, R2 is OCH₃, and R3 is SO₂NH₂, and R4 is H; or R1 is H, R2 is OCH₃, R3 is SO₂NH₂, and R4 is H; or R1 is CH₃, R2 is OCH₃, and R3 is SO₂NH(CH₃), and R4 is H;
if the kinase is CHK2, R1 is CH₃, R2 is H, and R3 is SO₂NH₂, and R4 is H; or R1 is CH₃, R2 is OCH₃, and R3 is SO₂NH₂, and R4 is H; or R1 is H, R2 is OCH₃, R3 is SO₂NH₂, and R4 is H;
if the kinase is ERBB4, R1 is CH₃, R2 is OCH₃, R3 is SO₂NH(CH₃), and R4 is H;
if the kinase is IRAK4, R1 is CH₃, R2 is H, and R3 is SO₂NH₂, and R4 is H; or R1 is CH₃, R2 is OCH₃, and R3 is SO₂NH₂, and R4 is H; or R1 is H, R2 is OCH₃, R3 is SO₂NH₂, and R4 is H; or R1 is CH₃, R2 is OCH₃, and R3 is SO₂NH(CH₃), and R4 is H; if the kinase is LCK, R1 is CH₃, R2 is H, and R3 is SO₂NH₂, and R4 is H; or R1 is CH₃, R2 is OCH₃, and R3 is SO₂NH(CH₃), and R4 is H;
if the kinase is MAP3K11, R1 is CH₃, R2 is H, and R3 is SO₂NH₂, and R4 is H ((2a21)); or R1 is CH₃, R2 is OCH₃, and R3 is SO₂NH₂, and R4 is H; or R1 is H, R2 is OCH₃, R3 is SO₂NH₂, and R4 is H;
if the kinase is RIPK2, R1 is H, R2 is OCH₃, R3 is SO₂NH₂, and R4 is H;
if the kinase is SGK1, R1 is H, R2 is OCH₃, R3 is SO₂NH₂, and R4 is H; or if the kinase is SYN aa1-635, R1 is H, R2 is OCH₃, R3 is SO₂NH₂, and R4 is H.

In another aspect of the invention, an engineered cell line for monitoring modulation of a tropomyosin receptor kinase (Trk) is provided, where the cell line is a transgenic population of rat adrenal pheochromocytoma (PC12) cells, wherein the PC12 cells express a tropomyosin receptor kinase selected from the group consisting of TrkA, TrkB and TrkC and include a first plasmid having a first promoter and encoding a GALA-DNA binding domain operatively connected to Elk1 transcription factor, the first plasmid further having a first antibiotic resistance gene in operative alignment with the promoter; and a second plasmid having, in operative alignment, a second promoter, a GAL4 upstream activation sequence (UAS), a luciferase reporter gene, and a second antibiotic resistance gene.

In a related aspect of the invention, a method of screening for inhibition of Trk in an engineered cell line is provided, were the method includes culturing the engineered cell line of claim in cell culture medium; exposing the cell line to an agonist of Trk; adding an inhibitor of Trk to the cell line; and monitoring the cell line for luciferase expression. In some embodiments the engineered cell line is cultured in a plurality of cell cultures and the inhibitor is added at different concentrations to different cell cultures, or different cell culture wells of the engineered cells. In such an embodiment, the method can also include determining an IC50 of the inhibitor.

### BRIEF DESCRIPTION OF THE DRAWINGS

FIG. 1 depicts an overview of the structures of compounds used in the data set, where A) provides exemplary oxindoles and aza-oxindoles, B) provides exemplary 3,5-disubstituted 7-azaindoles, and C) provides exemplary oxindole amides and ureas.

FIGS. 2A-2G is a table providing the ChEMBL ID, chemical structure, IC₅₀, and log IC₅₀ for 47 compounds tested for kinase inhibition.

FIG. 3 is a graphical depiction of the mechanism of luciferase induction in the PC-12/luc/Elk1 cell line, where upon binding of NGF to TrkA the Elk1 portion of expressed Elk1/GAL4-dbd protein becomes phosphorylated. Thereafter, the fusion protein binds GAL4 UAS and transcription of the luciferase gene is activated.

FIG. 4 is a graphical depiction of the Z′ factor of the assay system using PC-12/Elk1 cell line at different treatment times. PC-12/luc/Elk1 cells on a 96 well plate are treated for 6 h, 18 h or 24 h with either 50 ng/mL of NGF or 50 ng/mL of NFG together with 5 nM of AZ-23. Each graph point represents luciferase induction of one test well.

FIG. 5 is a flowchart showing the development of low-nanomolar TrkA inhibitors (IC₅₀'s are measured by cellular assays).

FIGS. 6A-6B are graphical characterizations of a QSAR model. In FIG. 6A, correlation of observed (biochemical assay) and predicted logiclC₅₀ for the training set and the external test set is shown. FIG. 6B shows Williams plot for verifying the applicability domain of the QSAR model, showing the relationship between standardized residuals (r′) and leverages (h). A more detailed colored view may be found in European Journal of Chemistry 121 (2016) 541-552.

FIG. 7 depicts the Z and E isomers of (Z)-34(5-methoxy-1H-indol-3-yl)methylene)-2-oxindole (2) and its derivatives in a table of observed and predicted IC₅₀ values.

FIG. 8 is a table depicting calculated IC₅₀ values on TrkA signaling measured in PC-12/luc/Elk1 cells.

FIGS. 9A-9C provide a table of data for Williams graph. Data set (T—training; E—external test), descriptors (D1—Lowest atomic state energy (AM1) for C atoms; D2—Lowest atomic state energy (AM1) for H atoms; D3—Max electrophilic reactivity index (AM1) for C atoms; D4—Molecular volume/XYZ box (AM1)) and parameters for the model (leverage value (h); standardized residual (r′); predicted (Pred.) and observed (Obs.) log IC₅₀; difference of predicted and observed values (Diff.); standard deviation (s²)).

FIG. 10 provides a Western blot image (A) and accompanying graph of Western blot results (B). In panel A) PC-12 or MG87/TrkB/luc/Elk1 cells were treated with 2a22, 2a25, and 4a22 at different concentrations concurrently with 50 ng/mL of neurotrophins NGF (for PC-12 cells) or BDNF (for MG87/TrkB/luc/Elk1 cells) for 5 min. Equal amount of lysates were analyzed using western blotting and the indicated antibodies. Neurotrophin alone was used as a negative control, 5 nM of AZ-23 as a positive control and “mock” corresponds to DMSO-treated cells. Representative blots of three independent experiments are shown. In panel (B) Quantified western blot signal ratios of phospho-Trk to total Trk were log-transformed, mean-centered, and autoscaled for ANOVA analysis. For graphical representation, the data was back-transformed and normalized to neurotrophin-treated cell lysate signals. Mean+/− SEM data is shown and asterisks indicate statistical significance according to Dunnett's post-hoc test comparing means with neurotrophin-treated cell lysate data mean (n=3, p<0.05).

FIG. 11 is a series of graphs depicting neuronal viability (A) or cellular ATP content (B) compared to compounds 2a22, 2a25 and 4a22. More specifically, in A) Primary cortical neurons were treated with 2a22, 2a25, and 4a22 at different concentrations for 24 h, after which the number of survived neurons were counted. The average of different measurements +/−SEM is shown (n=2 with 4-6 technical replicates). (B) MG87/TrkB/luc/Elk1 cells were treated with 2a22, 2a25, and 4a22 at different concentrations for 24 h and the cellular ATP content was quantified using CellTiter-Glo® Luminescent Cell Viability Assay. The average of different measurements +/−SEM is shown (n=2 with 4-6 technical replicates).

FIG. 12 depicts positions of tested proteins in the human kinase tree. The size of the circle depicts the level of inhibition by 4a22 at 100 nM concentration.

FIGS. 13A-13B provide tables summarizing data from biochemical profiling of 6 compounds at two concentrations, each against 48 protein kinases, average of duplicate measurements. Results are presented as percentage of residual activity. Boxed values identify residual activity less than or equal to 50%.

FIG. 14 is a graphical depiction showing the positioning of 4a22 and AZ-23 in the ATP binding site of TrkA.

FIG. 15 is a graphical depiction of the binding mode of 4a22 in the ATP-binding site of TrkA (PDB ID: 4AOJ). Hydrogen bonds and electrostatic interactions with corresponding lengths in Å are shown with dashed lines.

### DETAILED DESCRIPTION

Primary objects of the invention are to synthesize, identify, and characterize new low molecular weight antagonists of tropomyosin receptor kinase (Trk), preferably human Trk, for use as drug candidates for the treatment of Trk related disorders. Nonlimiting examples include compositions for the treatment of nervous system and other conditions associated with Trk, including but not limited to Trk-dependent molecular and physiological processes such as synaptic plasticity, neuronal differentiation and neurotrophin-induced neurotoxicity, inflammatory pain, cancer induced pain, and blocking the development of tumor cells. Among the cancers that may be treated with the compositions include pancreatic, prostate, breast, ovarian carcinoma, malignant melanomas, thyroid, and brain tumors.

The Trk antagonists of the invention share a similar indole-like basic structure; however, substituent substitution yielded compounds with markedly high activity and specificity for the Trk family. Among those tested, compounds 2a21, 2a22, 2a23, 2a25, 3a25 and 4a22 showed the highest inhibition of Trk activity. These compounds were also found to have no toxic or adverse effect on cortical neurons, PC-12 cells or dividing MG87/TRKB/luc/Elk1 cells, which indicates that these compounds are good candidates for therapeutic development.

In addition, biochemical profiling of a variety of low molecular weight compounds against 48 protein kinases and the cellular studies revealed modulation of targets other than the Trk family for some compounds, which may be due to a similar ATP binding pocket within these kinase domains. That is, while some compounds were highly selective for the Trk family and therefore are candidates for Trk-based therapeutics, other compounds effectively inhibited one or more remaining protein kinases in the panel. Accordingly, it is a broader object of the invention to provide low molecular weight kinase antagonists for use as drug candidates for the treatment of kinase related disorders, in particular in humans.

For clarity of disclosure, and not by way of limitation, the detailed description is divided into the subsections that follow. Further, unless defined otherwise, all technical and scientific terms used herein have the same meaning as is commonly understood by one of ordinary skill in the art to which this invention belongs. All patents, published patent applications and other publications referred to, are herein incorporated by reference in their entirety. If a definition set forth in this application is contrary to or otherwise inconsistent with a definition set forth in the patents, patent applications, and other publications that are incorporated by reference, the definition set forth in this application prevails over the definition that is incorporated by reference.

The invention is directed to kinase antagonists and their use for modulating kinase activity in cells, in particular, for inhibiting kinase activity for therapeutic treatment. The term “kinase antagonist” as used herein refers to a compound that acts against and blocks phosphorylation of a substrate by a kinase. A compound that blocks or inhibits kinase activity is also termed a “kinase inhibitor.” The term “inhibiting kinase activity” as used herein refers to reducing the rate of phosphorylation of a substrate by a kinase in response to exposure to a compound. The term “inhibiting kinase activity” does not require complete inhibition but instead requires at least partial reduction in activity. Kinase activity in the presence of an inhibitor is termed “residual activity” and can be determined by measuring the percent activity remaining after contacting the kinase with a kinase inhibitor compared to a suitable control.

In embodiments where the kinase is constitutively active, the invention includes a method of inhibiting kinase activity by providing a cell population expressing the kinase, and adding to the cell population a composition including the kinase antagonist in an amount sufficient to reduce the constitutive activity of the kinase. In some embodiments the cell is further exposed to a kinase agonist and kinase activity remains inhibited. As used herein the term “kinase agonist” refers a substance (either directly binding to a kinase domain or to other domains activating the kinase, such as adaptor-binding domain, for example) that increases its kinase activity (absent an inhibitor). As used herein the term “remains inhibited” refers to continued inhibition but does not require a same percent inhibition.

In embodiments where the kinase is activated by binding, such as by receptor-ligand binding, the invention also includes a method of inhibiting agonist (receptor ligand)-mediated kinase activity in a cell by providing a cell population expressing the kinase, and adding to the cell population a kinase antagonist in an amount sufficient to inhibit kinase activity compared to the activity of the kinase in the presence of the agonist alone, and optionally exposing the cell population to the agonist.

Cell populations provided in the methods include at least one cell but are typically a group of cells. These one or more cells may be of a same type or same cell lineage, or alternatively may be a mixture of different cell types or lineages. The cell populations have at least one cell expressing a functioning kinase, such as, but not limited to TrkA, TrkB or TrkC. The cell populations are nonlimiting with respect to species and source but are preferably mammalian cells and more preferably human cells and most preferably neural-like cells having the Trk signaling pathways intact. The cells may include one or more tumor cells, such as primary tumor cells. The cells may be cancer cells, optionally selected from stage I, stage II, stage III, or stage IV of a cancer. The cells may be cancerous cells of the pancreas, prostate, breast, ovary, thyroid, colon or brain. The cells may be malignant melanoma or a neuroblastoma. The cells may be primary cells isolated from a subject suffering from a medical condition that may benefit from kinase inhibition, such as inhibition of TrkA, TrkB or TrkC. The cells may be from one or more cell lines, such as a cancer cell line. The cells may express an endogenous kinase or may be engineered to display a recombinant or chimeric receptor with kinase activity.

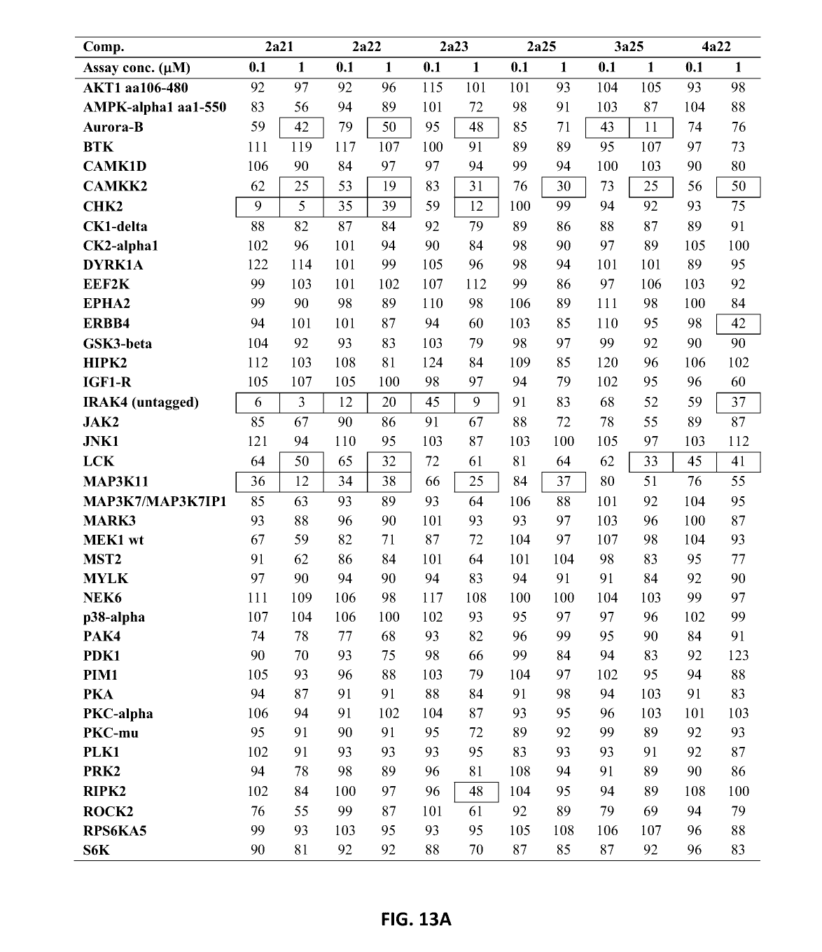

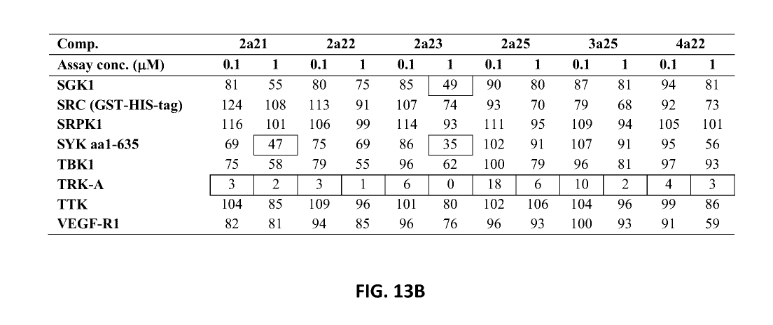

In methods where the cell population is exposed to an agonist, the agonist may be manually added, such as by adding a known agonist to the cell population in culture. Agonists for the kinases provided in FIGS. 13A-13B may be found in the literature. As nonlimiting examples, agonists for the Trk family may be neurotrophins, nerve growth factor (NGF), brain-derived neurotrophic factor (BDNF), neurotrophin 4 (NT4) and neurotrophin 3 (NT3). Alternatively, the agonist may be a known or unknown agonist in a biological system, such as an agonist circulating within the body of a subject in need of treatment.

Turning to the compositions themselves, the invention includes a compound of formula (I) or a pharmaceutically acceptable salt thereof, where formula (I) is

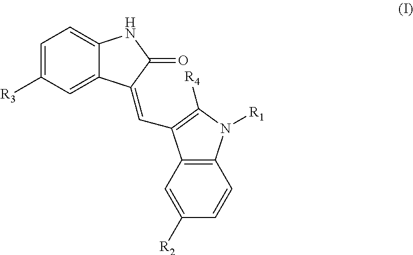

and where in formula (I), R1 is a lower alkyl; R2 is a hydroxy or lower alkoxy; R3 is SO₂N(CH₃)₂; and R4 is H. The lower alkyl is a straight or branched alkyl group having 1 to 6 carbon atoms, preferably 1-3 carbon atoms, more preferably 1 to 2 carbon atoms and most preferably a methyl. The lower alkoxy is a straight or branched alkoxy having 1 to 5 carbon atoms, preferably 1-3 carbon atoms, more preferably 1 carbon atom. In a preferred embodiment R1 is CH₃ and R2 is OCH₃. In another preferred embodiment, R1 is CH₃ and R2 is OH.

The invention also provides a composition, which includes a compound of formula (II) or a pharmaceutically acceptable salt thereof, where formula (II) is

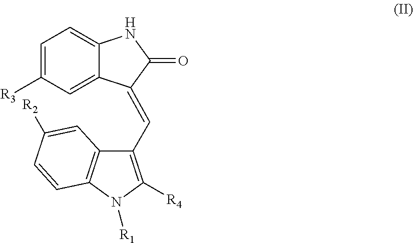

and where in formula (II), R1 is a lower alkyl; R2 is a hydroxy or lower alkoxy; R3 is SO₂N(CH₃)₂; and R4 is H. The lower alkyl is a straight or branched alkyl group having 1 to 6 carbon atoms, preferably 1-3 carbon atoms, more preferably 1 to 2 carbon atoms and most preferably a methyl. The lower alkoxy is a straight or branched alkoxy having 1 to 5 carbon atoms, preferably 1-3 carbon atoms, more preferably 1 carbon atom. In some embodiments, R1 is CH₃ and R2 is OCH₃. In other embodiments R1 is CH₃ and R2 is OH.

The invention also provides a kinase antagonist, where the kinase antagonist is compound 2a21. Compound 2a21 may be used to inhibit activity of one or more kinases, preferably human, selected from the group consisting of Aurora-B, CaMMK2, Chk2, Irak4, Lck, Map3K11, Syn aa1-635, TrkA, TrkB, and TrkC. An exemplary method includes a method of inhibiting kinase activity in a cell, where the cell expresses at least one kinase selected from the group consisting of Aurora-B, CaMMK2, Chk2, Irak4, Lck, Map3K11, Syn aa1-635, TrkA, TrkB, and TrkC; and adding to the cell population a composition including compound 2a21 in an amount sufficient to inhibit kinase activity; and optionally exposing the cell population to an agonist of the kinase. In some embodiments compound 2a21 is formulated as a pharmaceutical salt and/or is provided with a pharmaceutically acceptable carrier.

The invention also provides a kinase antagonist, where the kinase antagonist is compound 2a22. Compound 2a22 may be used to inhibit activity of one or more kinases, preferably human, selected from the group consisting of Aurora-B, CaMKK2, Chk2, Irak4, Lck, Map3K11, TrkA, TrkB, and TrkC. An exemplary method includes a method of inhibiting kinase activity in a cell, where the cell expresses at least one kinase selected from the group consisting of Aurora-B, CaMKK2, Chk2, Irak4, Lck, Map3K11, TrkA, TrkB, and TrkC; and adding to the cell population a composition including compound 2a22 in an amount sufficient to inhibit kinase activity; and optionally exposing the cell population to an agonist of the kinase. In some embodiments compound 2a22 is formulated as a pharmaceutical salt and/or is provided with a pharmaceutically acceptable carrier.

The invention also provides a kinase antagonist, where kinase antagonist is compound 2a23, also named (Z)-3((5-methoxy-1H-indol-3-yl)methylene)-2-oxindole-5-sulfonamide. Compound 2a23 may be used to inhibit activity of one or more kinases, preferably human, selected from the group consisting of Aurora-B, CaMKK2, Chk2, Irak4, Map3K11, Ripk2, Sgk1, Syn aa1-635, TrkA, TrkB, and TrkC. An exemplary method includes a method of inhibiting kinase activity in a cell, where the cell expresses at least one kinase selected from the group consisting of Aurora-B, CaMKK2, Chk2, Irak4, Map3K11, Ripk2, Sgk1, Syn aa1-635, TrkA, TrkB, and TrkC; and adding to the cell population a composition including compound 2a23 in an amount sufficient to inhibit kinase activity; and optionally exposing the cell population to an agonist of the kinase. In some embodiments compound 2a23 is formulated as a pharmaceutical salt and/or is provided with a pharmaceutically acceptable carrier.

The invention also provides a kinase antagonist, where the kinase antagonist is compound 2a25, also named (Z)-3-((5-methoxy-1-methyl-1H-indol-3-yl)methylene)-N,N-dimethyl-2-oxindole-5-sulfonamide. Compound 2a25 may be used to inhibit activity of one or more kinases, preferably human, selected from the group consisting of CaMKK2, Map3K11, TrkA, TrkB, and TrkC. An exemplary method includes a method of inhibiting kinase activity in a cell, where the cell expresses at least one kinase selected from the group consisting of CaMKK2, Map3K11, TrkA, TrkB, and TrkC; and adding to the cell population a composition including compound 2a25 in an amount sufficient to inhibit kinase activity; and optionally exposing the cell population to an agonist of the kinase. In some embodiments compound 2a25 is formulated as a pharmaceutical salt and/or is provided with a pharmaceutically acceptable carrier.

The invention also provides a kinase antagonist, where the kinase antagonist is compound 3a25, also named (Z)-3-((5-hydroxy-1-methyl-1H-indol-3-yl)methylene)-N,N-dimethyl-2-oxindole-5-sulfonamide. Compound 3a25 may be used to inhibit activity of one or more kinases, preferably human, selected from the group consisting of Aurora-B, CaMKK2, Lck, TrkA, TrkB, and TrkC. An exemplary method includes a method of inhibiting kinase activity in a cell, where the cell expresses at least one kinase selected from the group consisting of Aurora-B, CaMKK2, Lck, TrkA, TrkB, and TrkC; and adding to the cell population a composition of compound 3a25 in an amount sufficient to inhibit kinase activity; and optionally exposing the cell population to an agonist of the kinase. In some embodiments compound 3a25 is formulated as a pharmaceutical salt and/or is provided with a pharmaceutically acceptable carrier.

The invention also provides a kinase antagonist, where the kinase antagonist is compound 4a22, also named (Z)-3-((5-methoxy-1-methyl-1H-indol-3-yl)methylene)-N-methyl-2-oxindole-5-sulfonamide. Compound 4a22 may be used to inhibit activity of one or more kinases, preferably human, selected from the group consisting of CaMKK2, Erb-B4, Irak4, Lck, TrkA, TrkB, and TrkC. An exemplary method includes a method of inhibiting kinase activity in a cell, where the cell expresses at least one kinase selected from the group consisting of CaMKK2, Erb-B4, Irak4, Lck, TrkA, TrkB, and TrkC; and adding to the cell population a composition of compound 4a22 in an amount sufficient to inhibit kinase activity; and optionally exposing the cell population to an agonist of the kinase. In some embodiments compound 4a22 is formulated as a pharmaceutical salt and/or is provided with a pharmaceutically acceptable carrier.

In still other embodiments the invention provides a method of inhibiting kinase activity in a cell population, where the cell population expresses at least one kinase, preferably human, selected from the group consisting of TrkA, TrkB, and TrkC; and adding to the cell population a composition including a compound selected from one or more of 2a21, 2a22, 2a23, 2a25, 3a25 and 4a22 in an amount sufficient to inhibit kinase activity; and optionally exposing the cell population to an agonist of at least one of the at least one kinase. In some embodiments the compound is formulated as a pharmaceutical salt and/or is provided with a pharmaceutically acceptable carrier.

### Preparations for Therapeutic Use

In some embodiments, the compositions are the compounds themselves. However, in other embodiments the compositions are embodied as a pharmaceutically acceptable salts of the compounds, preferably for human. Such salts can include an acidic addition salt or a basic salt prepared by causing one or more pharmaceutically acceptable acid or basic compounds to act on any of the compounds of formula (I) or formula (II).

Examples of acid addition salts are salts of the compounds of formula (I) or (II) having a basic group, especially an amino group, or a mono- or di-lower alkylamino group with an acid, such as an inorganic acid including but not limited to hydrochloric; acid, sulfuric acid, phosphoric acid, and hydrobromic add, or an organic acid including but not limited to oxalic acid, maleic acid, fumaric acid, malic acid, tartaric acid, citric acid, benzoic acid, acetic acid, p-toluenesulfonic acid, and ethanesulfonic acid. Examples of basic salts include salts of the compounds of the formula (I) or formula (II) having an acidic group, especially a carboxyl group with a base; e.g., salts of alkali metals such as sodium, or potassium or salts of alkaline earth metals such as magnesium or calcium, and further include organic salts of the compounds of the formula (I) or formula (II) with amines such as ammonia, methylamine, dimethylamine, piperidine, cyclohexylamine; or triethylamine.

The salts of the compounds can be easily produced by reacting each free compound with any of the above-exemplified acids or basic compounds by a conventional method or as known in the pharmaceutical arts.

For use as medicaments, the compounds can be made into various pharmaceutical dosage forms according to a preventive or therapeutic purpose. Examples of pharmaceutical dosage forms are oral preparations, injections, suppositories, ointments, plasters and so on. Such preparations can be formulated in a manner already known and conventional to those skilled in the art to which the invention belongs.

The amount of the compound to be incorporated into each of the unit dosage forms varies with the medical condition of the patient or with the type of the preparations. The preferable amount per dosage unit is about 1 to about 1,000 mg for oral preparations, about 0.1 to about 500 mg for injections, or about 5 to about 1,000 mg for suppositories. The dosage per day of the drug in the above dosage forms can be variable with the symptoms, body weight, age, sex and other factors of the patient, but usually ranges from about 0.1 to about 5,000 mg, preferably from about 1 to about 1,000 mg: for human adult per day. The preparation is preferably administered in a single dose or in two to four divided doses.

For the formulation of solid preparations for oral administration; an excipient and, when required, a binder, disintegrator, lubricant, coloring agent, corrigent, flavor, etc. are added to the compound, and then a preparation is formulated in a conventional way as tablets, coated tablets, granules, powders, capsules or others. Such additives are those already known in the art, and useful examples are excipients such as lactose, sucrose, sodium chloride, glucose, starch, calcium carbonate, kaolin; microcrystalline cellulose and silicic acid; binders such as water; ethanol, propanol, simple syrup, glucose solution, starch solution, gelatin solution, carboxymethyl cellulose, hydroxypropyl cellulose, hydroxypropyl starch; methyl cellulose; ethyl cellulose, shellac, calcium phosphate and polyvinyl pyrrolidone; disintegrators such as dried starch, sodium alginate, agar powder, sodium hydrogencarbonate, calcium carbonate, sodium lauryl sulfate; stearic acid monoglyceride and lactose; lubricants such as purified talc, stearic acid salt, borax and polyethylene glycol; corrigents such as sucrose, bitter orange peel, citric acid and tartaric acid, etc.

As a nonlimiting embodiment; a tablet can be prepared in a convention manner using the following components of the proportions indicated below.

| | | |
|---|---|---|
| Compound 2a21 | 100 | mg |
| Lactose | 47 | mg |
| Corn starch | 50 | mg |
| Microcrystalline cellulose | 65 | mg |
| Talc | 2 | mg |
| Magnesium stearate | 2 | mg |
| Ethyl cellulose | 30 | mg |
| Unsaturated fatty acid glyceride | 4 | mg |
| Per tablet | 300 | mg |

As a nonlimiting embodiment granules can be prepared in a convention manner using the following components of the proportions indicated below.

| | | |
|---|---|---|
| Compound 2a22 | 200 | mg |
| Mannitol | 540 | mg |
| Corn starch | 100 | mg |
| Crystalline cellulose | 100 | mg |
| Hydroxypropyl cellulose | 50 | mg |
| Talc | 10 | mg |
| Per wrapper | 1000 | mg |

As a nonlimiting embodiment capsules can be prepared in a convention manner using the following components of the proportions indicated below.

| | | |
|---|---|---|
| Compound 2a23 | 300 | mg |
| Lactose | 50 | mg |
| Corn starch | 47 | mg |
| Crystalline cellulose | 50 | mg |
| Hydroxypropyl cellulose | 50 | mg |
| Talc | 2 | mg |
| Magnesium stearate | 1 | mg |
| Per capsule | 500 | mg |

For the formulation of liquid preparations for oral administration, a corrigent, buffer, stabilizer, flavor, etc. can be added to the compound, and the mixture can be formulated in a conventional way into an oral liquid preparation, syrup, elixir or the like. Examples of useful corrigents are those exemplified above. Examples of buffers are sodium citrate, etc. Examples of stabilizers are tragacanth, gum arabic, gelatin, etc. As a nonlimiting embodiment syrups can be prepared in a convention manner using the following components of the proportions indicated below.

| Compound 3a25 | 500 | mg |
|---|---|---|
| Sucrose | 30 | mg |
| Gelatin solution | Suitable amount | |
| Flavoring/Coloring | Suitable amount | |
| Purified water | q.s | |
| Total | 500 | mL |

Injections can be prepared as a subcutaneous, intramuscular or intravenous injection in a conventional way by adding to the compound a pH adjusting agent, buffer, stabilizer, isotonic agent, local anesthetic, etc. Examples of pH adjusting agents and buffers are sodium citrate, sodium acetate, sodium phosphate, etc. Examples of stabilizers are sodium pyrosulfite, thioglycolic acid, thiolactic acid, etc. Examples of local anesthetics are procaine hydrochloride, lidocaine hydrochloride, etc. Examples of isotonic agents are sodium chloride, glucose, etc. As a nonlimiting embodiment, injection formulations can be prepared in a convention manner using the following components of the proportions indicated below.

| Compound 4a22 | 50 | mg |
|---|---|---|
| pH buffered saline | q.s. | |
| Total per ampule | 1 | mL |

Suppositories can be prepared in a usual manner by adding to the compound a pharmaceutically acceptable carrier already known in the art, such as polyethylene glycols, lanolin, cacao fat and oil, fatty acid triglycerides and, if desired, a surfactant such as TWEEN. As a nonlimiting embodiment, suppositories can be prepared in a convention manner using the following components of the proportions indicated below.

| | | |
|---|---|---|
| Compound 2a23 | 100 | mg |
| Triglyceride of saturated fatty acid | 1400 | mg |
| Total per suppository | 1500 | mg |

For the preparation of ointments, a base, a stabilizer, a humectant, a preservative and the like commonly used in the art are used as required. These additives together with the compound are formulated into ointments by conventional methods. Useful examples of the base include, for example, liquid paraffin, white petrolatum, bleached beeswax, octyl dodecyl alcohol, paraffin, etc. As preservatives, there can be mentioned methyl para-hydroxybenzoate, ethyl para-hydroxybenzoate, para-hydroxy propyl benzoate, etc.

For the preparation of plasters, ointment, cream, gel or paste the compound is applied to a substrate commonly employed in the art in a conventional manner. Suitable examples of substrates are woven or non-woven fabrics of cotton, rayon, chemical fibers or the like and films or foamed sheets of soft vinyl chloride, polyethylene, polyurethane or the like.

### Model Development

The technical approach used to identify the kinase antagonists relied on computational molecular modeling (quantitative structure-activity relationship (QSAR) and fragment-based QSAR), chemical synthesis, and testing the compounds biochemically as well as in cellular assays.

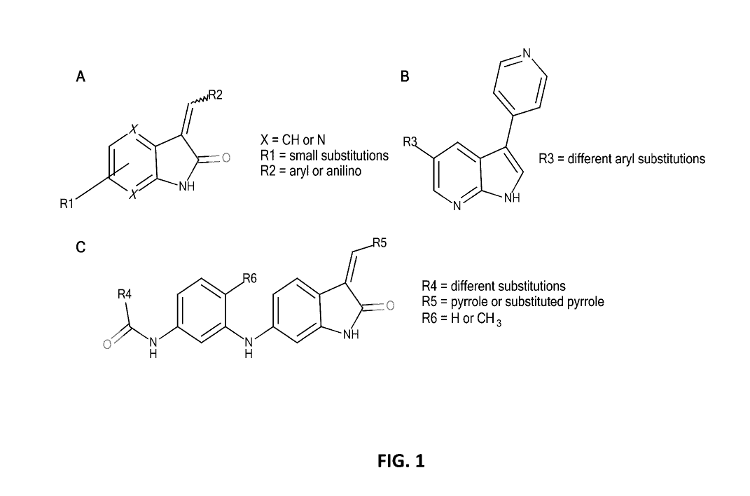

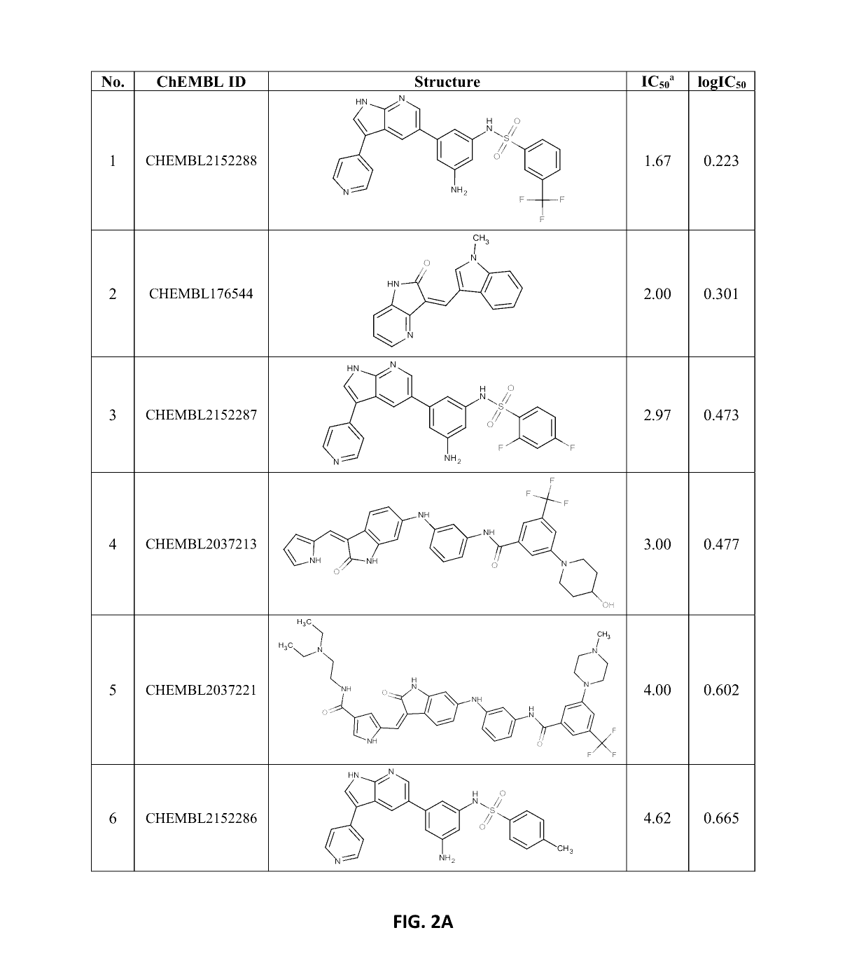

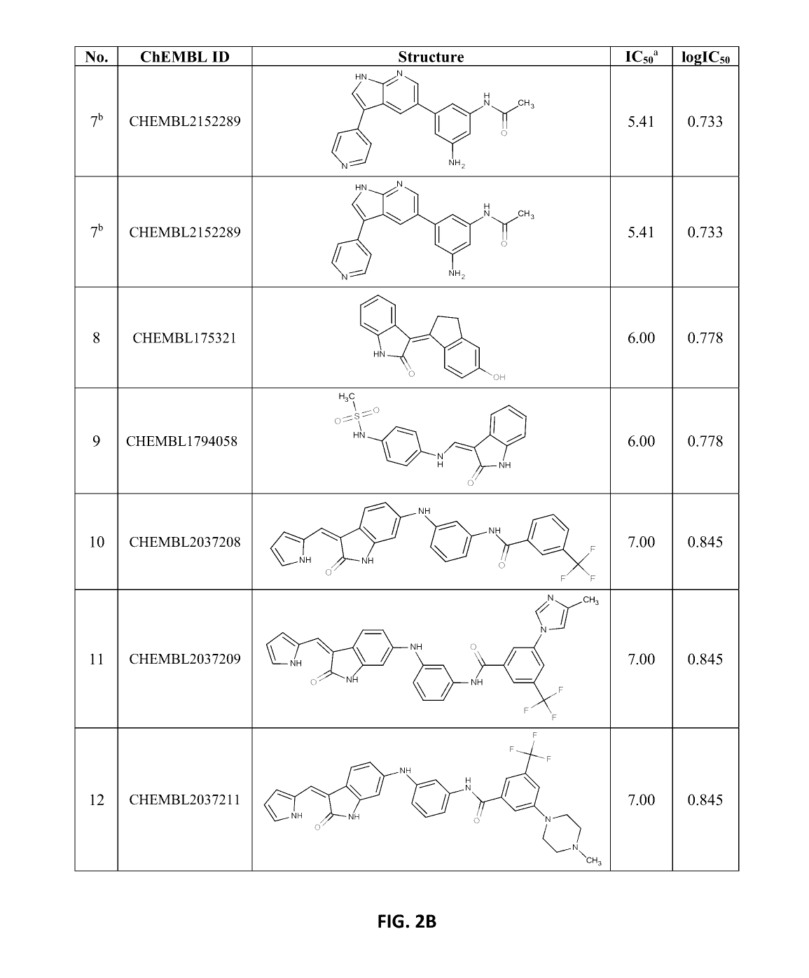

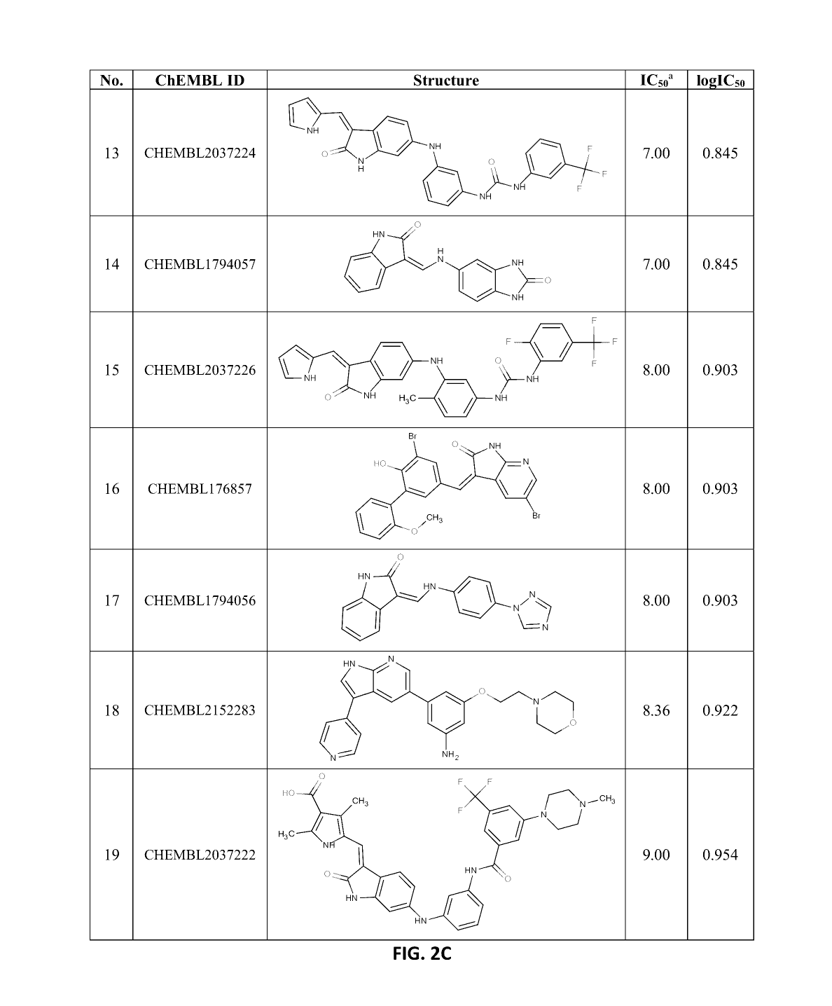

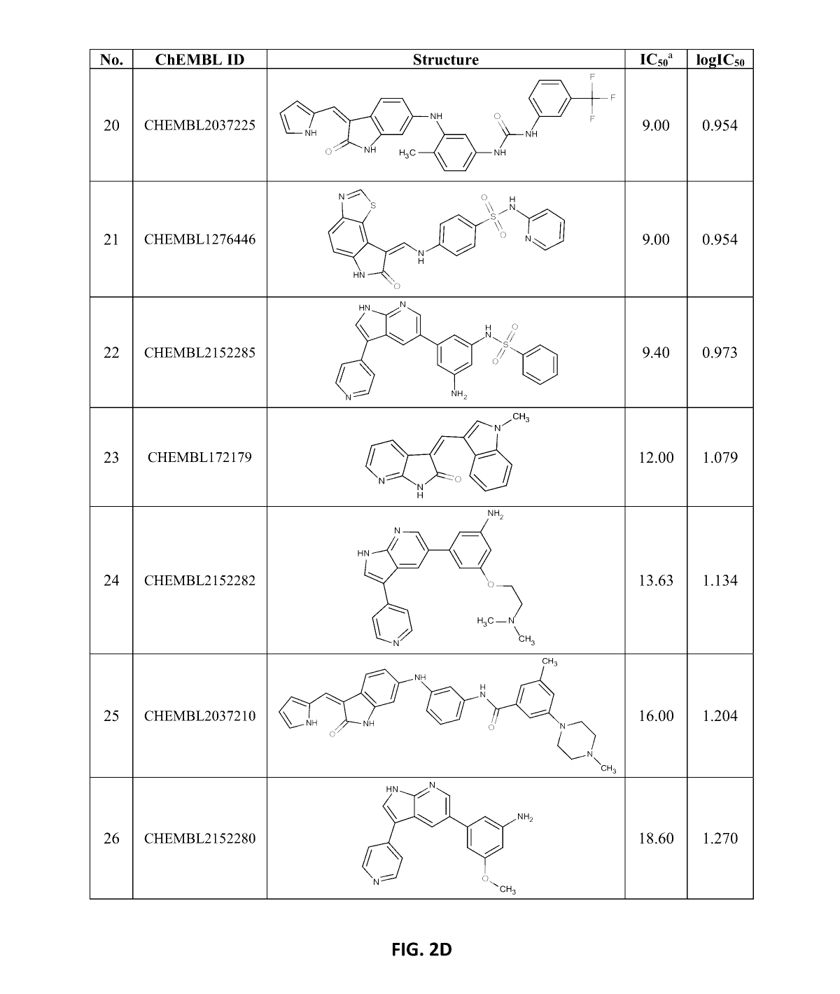

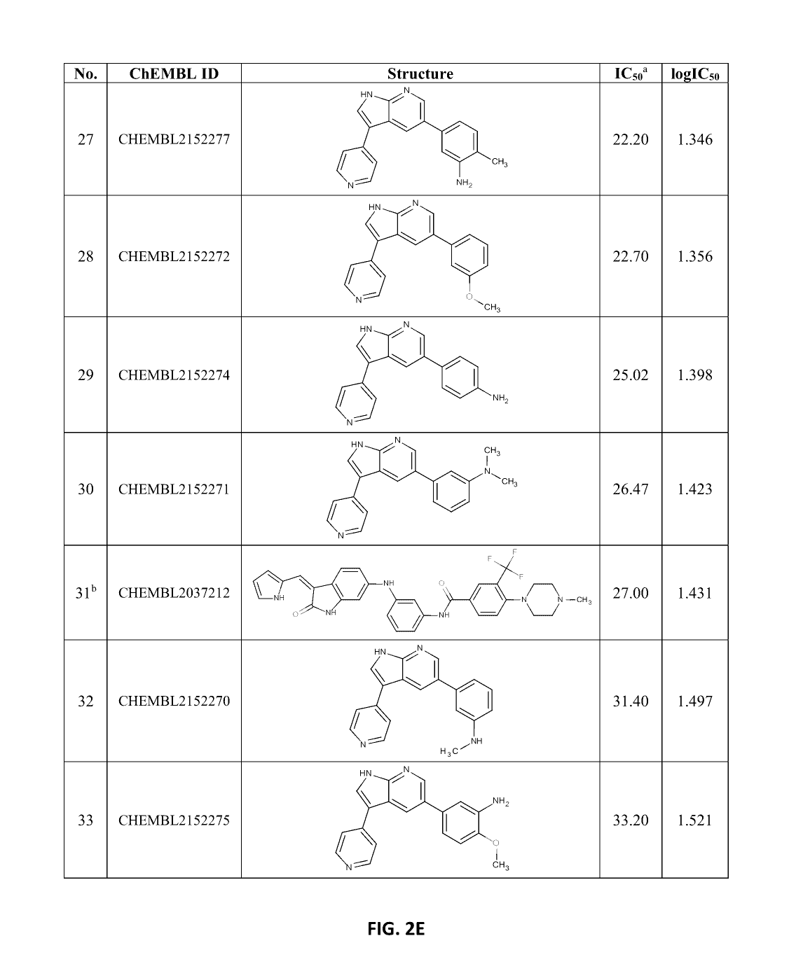

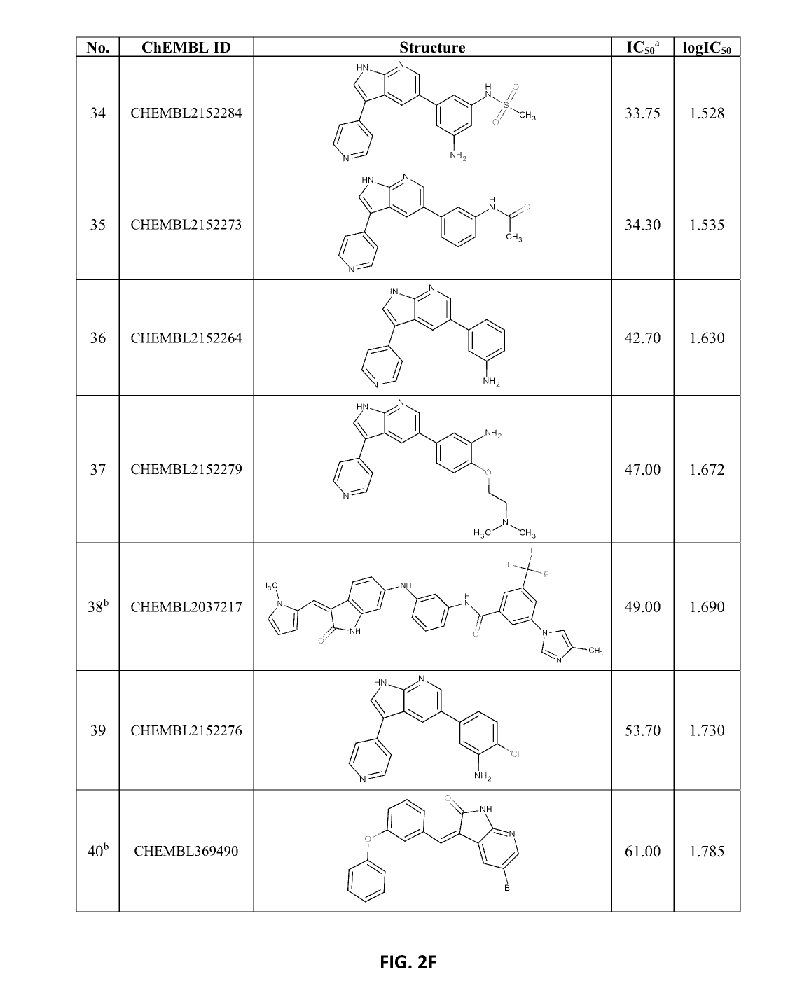

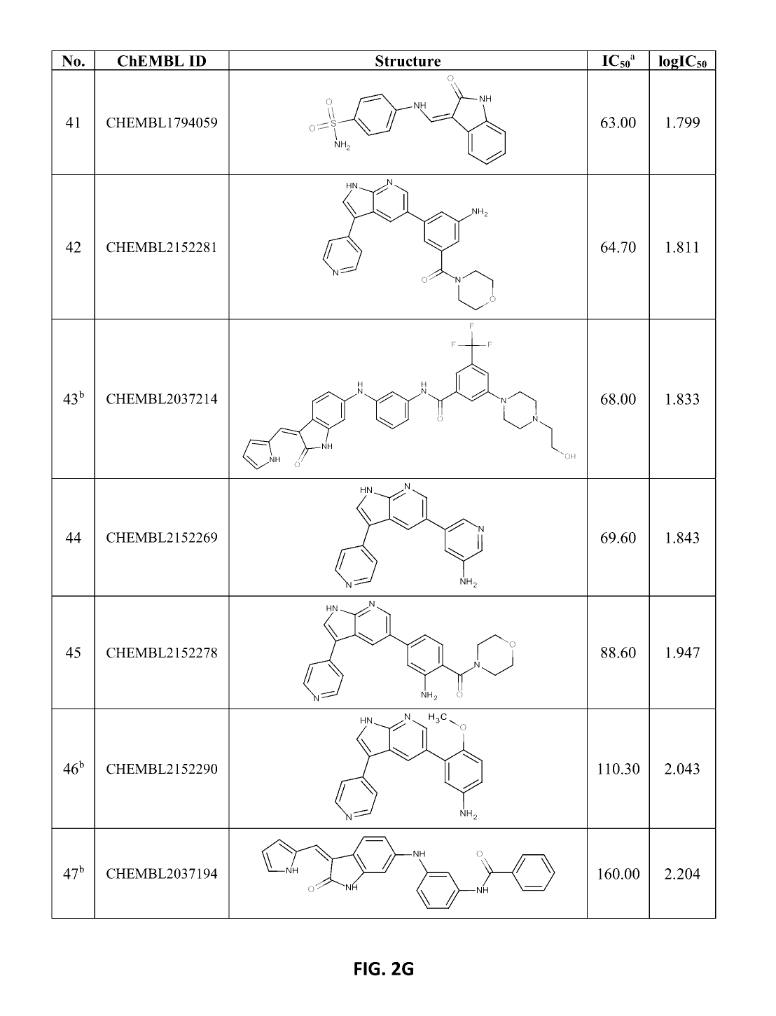

The data set obtained consisted of 47 indoles, which included (A) oxindoles and aza-oxindoles, (B) 3,5-disubstituted 7-azaindoles, and (C) oxindole amides and ureas (FIG. 1 and FIGS. 2A-2G). Using the QSARModel program, several multiple linear regression (MLR) models were developed:

$$P = {P}_{0} + {\sum }_{i = 1}^{n} {a}_{i} {D}_{i} \qquad ( Equation 1 )$$

Equation (1) correlates the studied property/activity P (P₀—intercept) (in this case log IC₅₀) with a certain number n of molecular descriptors Dᵢ weighted by the regression coefficients αᵢ. Up to seven-parameters models were composed. As the compounds belong to three different structural classes and the corresponding biochemical assays differ from each other, the data set was investigated for outliers. At first 4 outliers (compounds 38, 40, 43, and 46 in FIGS. 2A-2G), thereafter 3 outliers (compounds 7, 31, and 47 in FIGS. 2A-2G) were identified by modified leverage analyses, i.e. compounds with large deviations from the model s² were removed:

(log IC_{50(predicted)}−log IC_{50(observed)})²>s² (Equation 2)

The final best MLR model of four descriptors possessed statistical characteristics is shown in TABLE 1. The coefficient of determination (Pearson's squared correlation coefficient) is R²=0.770 for the data set of 40 compounds.

TABLE 1
The QSAR BMLR Model, its Statistics and Validation

Statistics
N = 40 n = 4^{[b]} R² = 0.770 R²_{cv} = 0.708 F = 29.316 s² = 0.225
Equation and descriptors
logIC₅₀ = 71.968 + 0.618 × D1 + 0.648 × D2 − 54.116 × D3 −
2.291 × D4 (Equation 3)
D1 - Lowest atomic state energy (AM1) for C atoms
D2 - Lowest atomic state energy (AM1) for H atoms
D3 - Max electrophilic reactivity index (AM1) for C atoms
D4 - Molecular volume/XYZ box (AM1)
Validation
ABC Cross-validation results
AB: R² = 0.780; R²_{cv} = 0.696
BC: R² = 0.720; R²_{cv} = 0.436
AC: R² = 0.822; R²_{cv} = 0.735
R²_{avg} = 0.744; R²_{cv}_{_{—}}_{avg} = 0.622

In TABLE 1, R² is Pearson's squared correlation coefficient, R²_{cv} is squared correlation coefficient for leave-one-out validation, F is Fisher statistics, s² is squared standard deviation of the model. The four-parameter model was chosen because of the breaking point in the graph n vs R² and R²_{cv}.

An ABC validation test was applied to estimate the predictivity of Equation (1), taking into account the property data distribution. The ABC method sorts the in an ascending order according to the observed (experimental) values and three subsets (A, B, C) are formed: the 1st, 4th, 7th, etc. data points comprised the first subset (A), the 2nd, 5th, 8th, etc. comprised the second subset (B), and the 3rd, 6th, 9th, etc. comprised the third subset (C). Then three training sets were prepared as the combinations of any two subsets. Subsequently, the tested MLR model was rebuilt for each of the training sets, (AB, AC, and BC), with the same descriptors but with optimized regression coefficients. Further, these three models AB, AC, and BC were used to predict the property values for the C, B, and A subsets, respectively. The prediction was assessed based on the coefficient of determination R² between the predicted and observed property values. The final result was estimated by the averaged squared correlation coefficient by the three “external” sets C, B, and A. As regarding this ABC validation, the averaged R² is close to the R² of model, which is good for prediction purposes. In addition to the ABC validation, the standard leave-one-out cross-validation (R²_{cv}) for the QSAR model resulted in R²_{cv}=0.708.

The descriptors appearing in the QSAR model (TABLE 1) are related to the stability, energy partition, and shape of the molecules. The quantum-chemical descriptors, the lowest atomic state energy (AM1) for C atoms and the lowest atomic state energy (AM1) for H atoms are related to the ground states of these atoms in the molecule. The lower the energy, the more stable is the atomic system and, thus, the more stable is the molecule with large C and H content. Besides, atomic state energies in QSAR models can be related to the change in the ligand electronic structure, steric hindrance, and the corresponding energetic effects in binding to the receptor. The quantum-chemical descriptor, the maximum electrophilic reactivity index (AM1) for C atoms comes from the LUMO coefficients and estimates the relative reactivity of the atoms within the molecule for a given series of compounds and is related to the activation energy of the corresponding chemical reaction. Since most atoms are the C atoms in the approach, this descriptor can be responsible for the reactivity of compounds. The molecular volume/XYZ box (AM1) is the geometrical descriptor that describes the bulk related properties and, by normalizing the descriptor with a unit box, shows how compact the molecule is.

### Generation of a Stable Cell-Line to Monitor Trk Activity

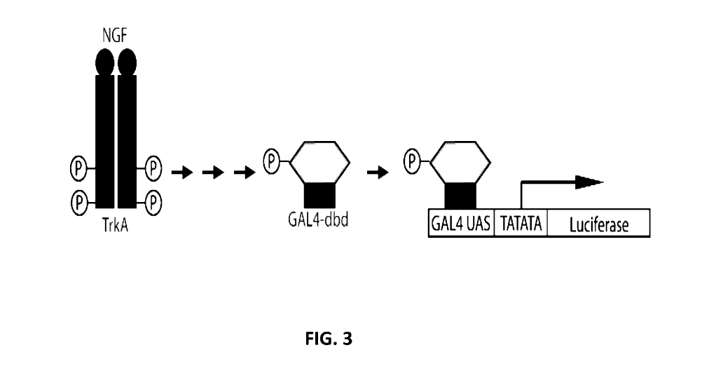

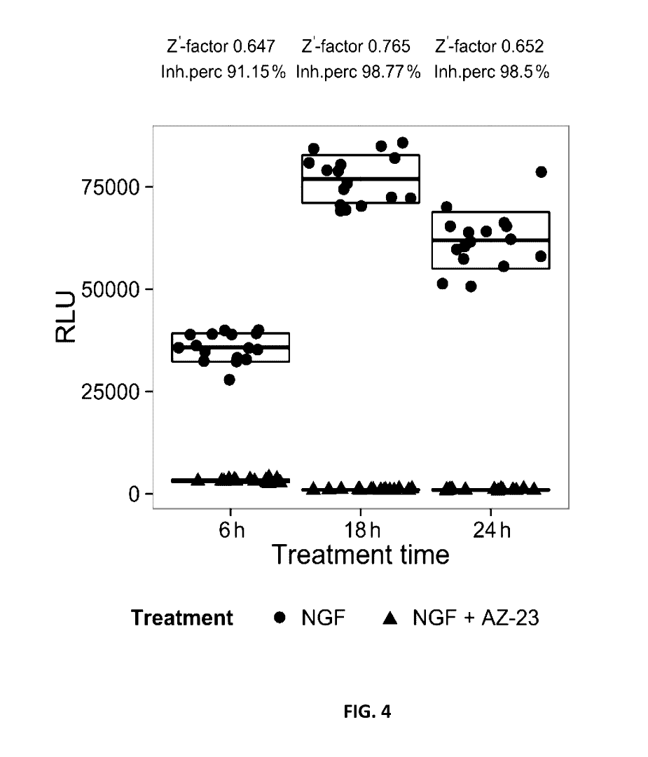

A stably transfected cell line was generated in order to assess the capability of the compounds to inhibit the activation of TrkA receptor in the natural (neural) cellular context. Once the endogenic TrkA of the PC-12/luc/Elk1 cell line becomes activated, the downstream events result in phosphorylation of Elk1, the GAL4-dbd fused to Elk1 binds then to GAL4 UAS and activates the transcription of luciferase (FIG. 3). To address the suitability of the generated system for screening application, the Z′-factor was determined (see also Examples section). The Z′-factor for this assay based on the negative control NGF and the known TrkA inhibitor AZ-23 as the positive control was measured to be 0.647 after 6 h, 0.765 after 18 h, and 0.652 after 24 h of treatment (FIG. 4). Z′-factor value above 0.5 is considered an indication of a high quality assay. The timeframe of 18 hours with the highest Z′-factor value was chosen for subsequent experiments.

### Characterization of New Potent Trk Inhibitors

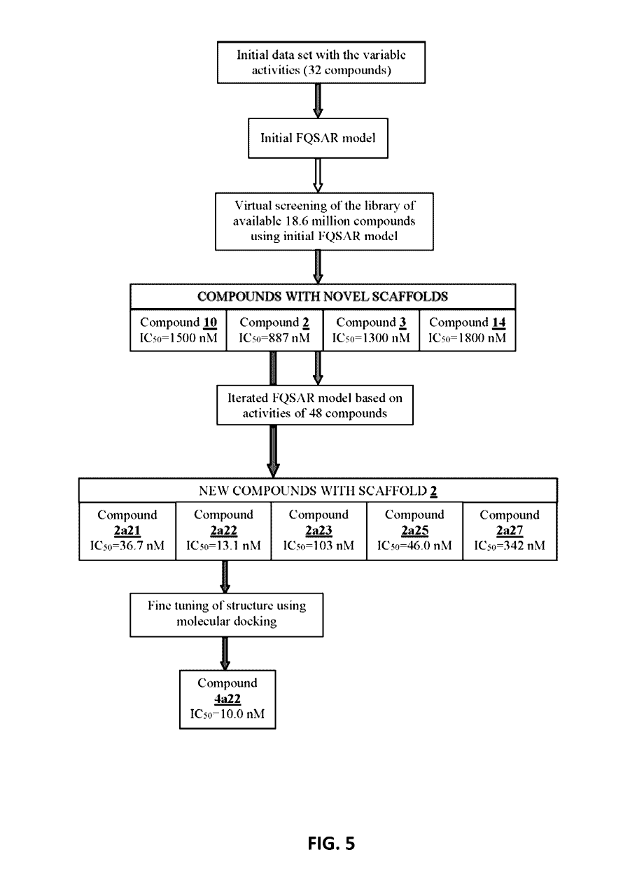

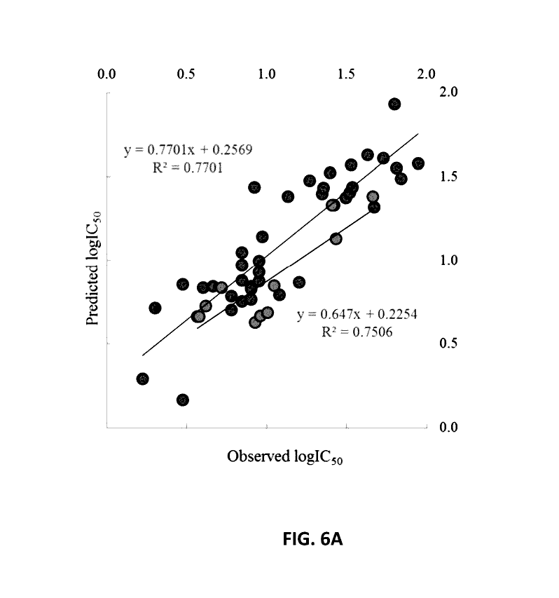

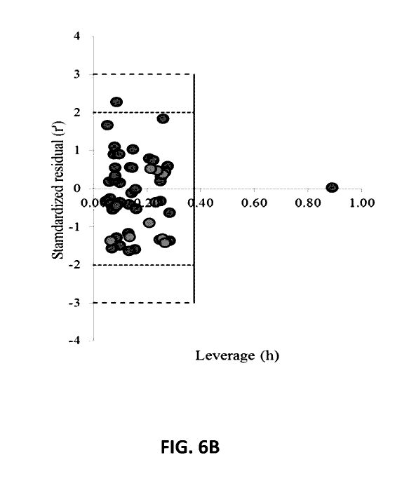

TrkA was selected as a representative of Trk receptors for virtual screening of compounds targeting the ATP-binding pocket. At first, virtual screening for new scaffolds using Tanimoto similarity was made by search in ZINC, MolPort, and ChEMBL databases. According to the obtained results, measurements by cellular assays were carried out for four compounds. In the next step, (Z)-3-((5-methoxy-1H-indol-3-yl)methylene)-2-oxindole (2; code numbers used are based on our virtual screening) and its 47 derivatives were found as potential TrkA inhibitors (see FIG. 5). The respective descriptors obtained by the FQSARModel program (see Examples section) were used in the full-molecular QSAR model (FIG. 6A) to predict their IC₅₀ values according to Equation 3 (see TABLE 1).

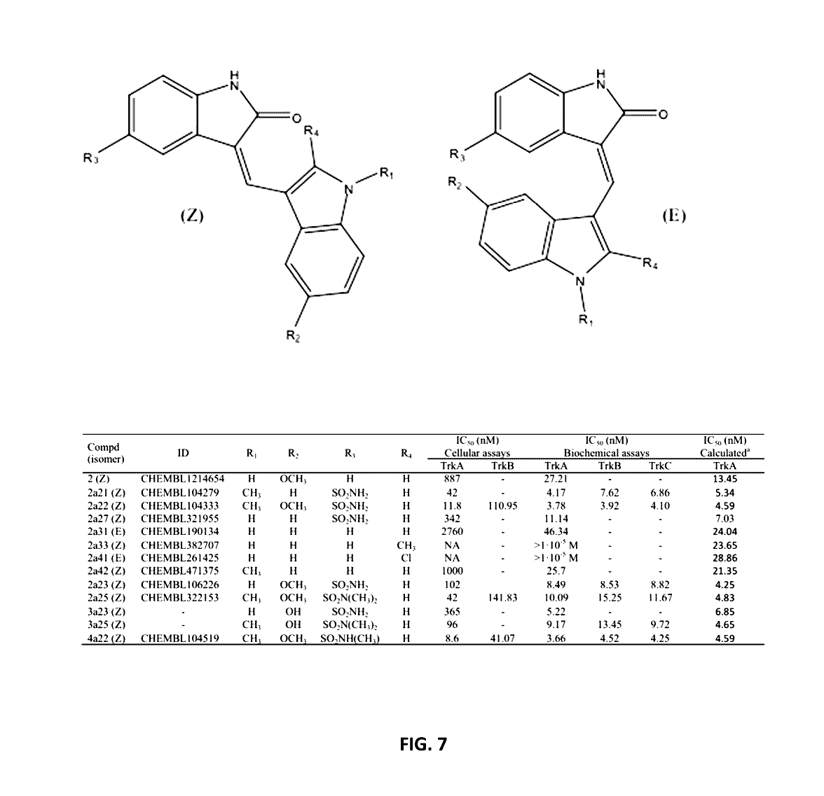

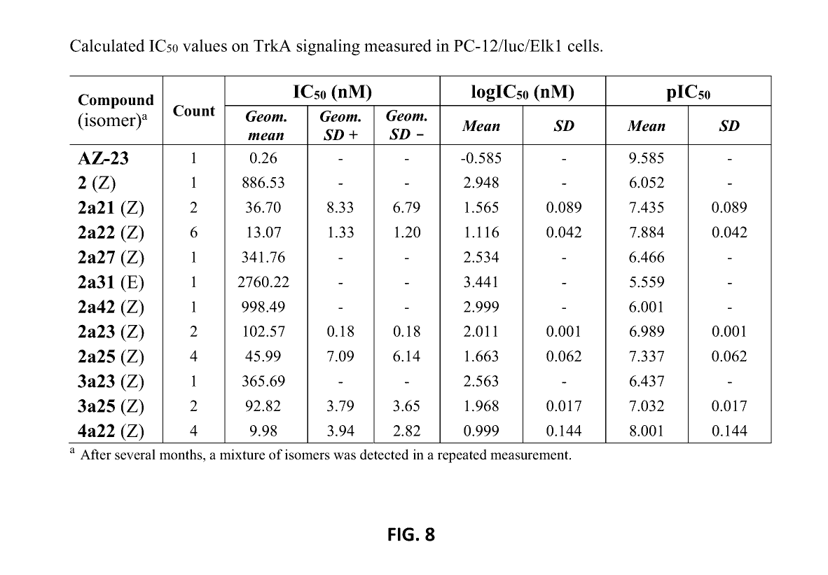

Commercially available compounds were purchased (FIGS. 2A-2G) and measured for the inhibitory activity using cellular and biochemical assays (FIG. 7; detailed data of cellular assays is presented in FIG. 8). The best experimental result from the first series (2-2a42) was obtained for compound 2a22. According to the structure-activity relationships (SAR), the sulfonamide has the strongest influence on the inhibitory activity among the pharmacophores. Compounds without this functionality show somewhat less activity (2, 2a31, 2a42) or are mostly inactive (2a33, 2a41).

Since the discovery of Prontosil prodrug against bacterial infection, sulfonamides have been widely exploited in pharmacy. Apart from the bacterial infections, sulfonamides are used for numerous other clinical indications. Typically, they have been applied as thiazide and loop diuretics. The sulfonamide COX-1/COX-2 and COX-2 inhibitors such as Celecoxib, SC-558, Rofecoxib, and DuP-697 have been prescribed against inflammation, pain, and cancers. Another area of sulfonamide drugs includes HIV protease and reverse transcriptase inhibitors.

Therefore, sulfonamides are a well-studied class of compounds in medicinal chemistry, with ample data available on their pharmacokinetic, pharmacodynamics, and ADME/Tox properties. Some sulfonamides are characterized by rapid oral absorption and their metabolic pathways are well understood. The available large data enables robust optimization of the compound's structure to find the best drug-like properties.

Our interest was turned to possible sulfonamide derivatives of indoles. Based on compound 2a22, five compounds were synthesized (2a23, 2a25, 3a23, 3a25, 4a22), combining the substitutions at the indole and/or oxindole rings. The cellular and biochemical assays showed contradictory results (see FIG. 7), thus, the following discussion is based on the biochemical assays as more directly related to TrkA kinase activity. Besides, due to the modeling data set, the predicted IC₅₀ values correspond to the biochemical assays. The substitution of methoxy group by hydroxyl group in indole (R₂) increased the activity (2a23 and 2a25 vs. 3a23 and 3a25, respectively) but N-methylation in indole (R₁) and N,N-dimethylation in sulfonamide of oxindole (R3) reduced the TrkA inhibiting effect (2a25 and 3a25 vs. 2a23 and 3a23). Nevertheless, the biochemically measured IC₅₀ values were similar for these four compounds. The N-methylation in compounds has insignificant effect on their inhibitory activity. Among the studied compounds, the best TrkA inhibitor according to both the cellular (10.0 nM) and biochemical (3.7 nM) assays was 4a22 that has N-methylated indole and N-methylated sulfonamide functionalities. Still, its activity was comparable to compound 2a22 that has only indole N-methylated. Thus, N-methylation in indole could have somewhat stronger effect (2a21 vs. 2a27).

Additionally, the strongest inhibitor 4a22 was selected to identify its binding mode and interactions in the ATP-binding site of TrkA. The molecular docking study is described below.

In general, the IC₅₀ values obtained with biochemical assays were lower compared to cellular assays. However, the relative activity remained similar with the strongest inhibitors being 2a22 and 4a22 and the IC₅₀'s above the measurement range for 2a33 and 2a41. The differences in measurements of cellular and biochemical assays could have resulted from the solubility of the compounds. However, the aqueous solubility at room temperature of compounds 2a22, 2a25, and 4a22 did not differ significantly, it was determined as 9.7±0.3, 7.8±0.2, and 3.3±0.3 μM (i.e. 3.7±0.1, 3.2±0.1, and 1.3±0.1 μg/mL), respectively, showing that all compounds were soluble at the tested concentrations. It is also possible that the differences in the IC₅₀ values measured by cellular and biochemical assays could yield from other reasons, for example specificity among intracellular interaction partners and cell permeability properties of the compounds. In addition, six compounds with the highest inhibition rates against TrkA were characterized biochemically against TrkB and TrkC. The results show that these compounds exhibit no significant selectivity of TrkA over TrkB and TrkC (TABLE 2). The tendency of correlation between functional groups and compound activity to inhibit TrkB and TrkC is similar to the correlation previously described for TrkA measurements.

### Comparison of Biochemical and Predicted IC₅₀ Values

In this study, the training set was constructed using the modeling data set and included 40 compounds from the final QSAR model. The external test set was based on compound 2 and its 10 derivatives with measured biochemical values. The correlation between biochemically measured and predicted IC₅₀'s for the both sets is rather satisfactory (R²=0.770 and 0.751, respectively; FIG. 6A).

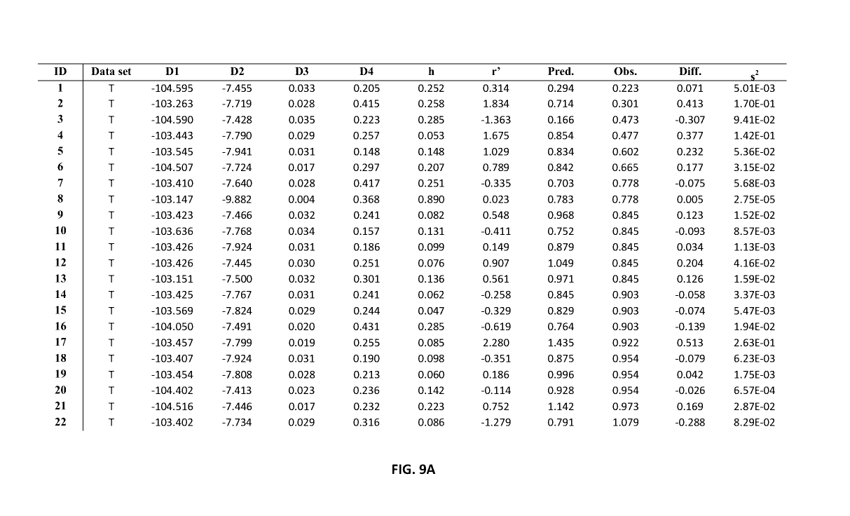

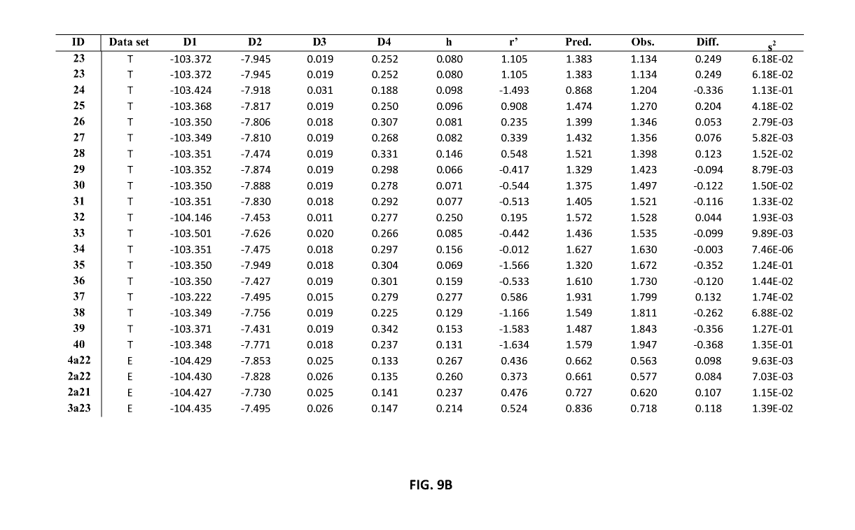

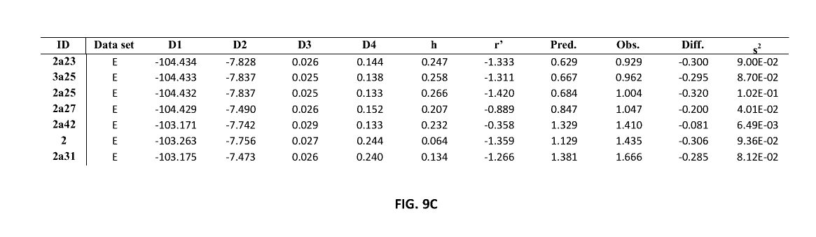

Williams graph (FIG. 6B) illustrates which points deviate because of descriptors and which points because of experimental values. According to leverage value h of descriptors, one compound (entry 9 in FIGS. 2A-2G) deviates significantly because of its extreme values of descriptors D2 and D3 (entry 8 in FIGS. 9A-9C). Other compounds stay within the critical area determined by a vertical line at 0.375. Compounds of the external test set belong to the applicability domain, i.e. they are structurally similar to the training set. According to the standardized residual r′ for biochemically measured data, all compounds deviate more than 2.5 units from the correlation line are considered to be strong deviations. One compound (entry 18 and 17 in FIGS. 2A-2G and FIGS. 9A-9C, respectively) from the training set is quite close to this critical point. In case of the external test set, the results are somewhat underestimated, which is probably due to the different method to predict IC₅₀'s for the training set (obtained directly by the QSARModel) and the external test set (descriptors obtained by FQSARModel were used in the full-molecular best MLR QSAR model).

Compounds 2a22, 2a25, and 4a22 are Potent Inhibitors of Both TrkA and TrkB in Cellular Context

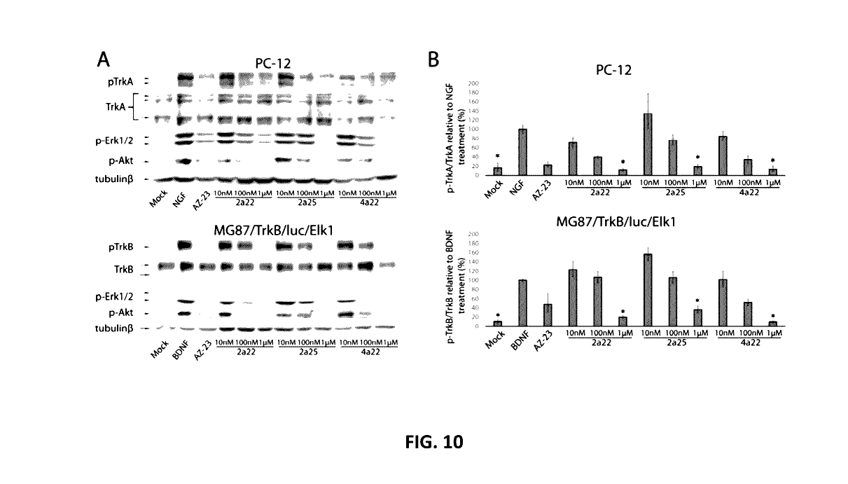

According to experimental results with the PC-12/luc/Elk1 cell line, three compounds—2a22, 2a25, and 4a22 were selected for further testing by western blot using antibodies specific for phosphorylated TrkA, TrkB, and their downstream kinases. Compounds 2a22 and 4a22 were two of the most efficient new inhibitors tested. Compound 2a25 was chosen, as it has been shown not to suppress Syk activity, whereas 2a21, 2a22, 4a22 as well as 2a23 were reported as Syk inhibitors. The compounds were tested on PC-12 and MG87/TrkB/luc/Elk1 cells that express TrkA and TrkB, respectively. The cells were treated concurrently with NGF or BDNF and the compounds at three different concentrations. The levels of phosphorylated kinases were assessed using respective antibodies and further quantification of western blot signals. The phosphorylation of TrkA and TrkB was effectively inhibited by all of the tested compounds with statistically significant reduction in activity at concentration of 1 μM The phosphorylation of downstream kinases Akt and Erk1/2 was observed to be diminished likewise compared to the positive control NGF or BDNF (FIG. 10). These results are consistent with the biochemical IC₅₀ results obtained for the Trk family kinases, which showed no selectivity of these compounds in inhibiting TrkA over TrkB and TrkC (FIG. 7). However, luciferase assays performed with PC-12/luc/Elk1 and MG87/TrkB/luc/Elk1 cell lines indicate that the given compounds are more potent inhibitors of TrkA than of TrkB in given cellular contexts (FIG. 7). This is most probably caused by the use of different cell lines to assay the inhibition of TrkA (PC-12/luc/Elk1) and TrkB (MG87/TrkB/luc/Elk1) that possibly can have different off-target proteins for the tested compounds, as well as slightly dissimilar membrane properties that can affect the influx of these chemicals. Additionally, in these two distinct cellular systems the activity of the Trk proteins may be incomparable due to their different concentration or interaction partners that can affect their behavior. For this reason, most probably there is no big selectivity of these compounds for the Trk kinases which is expected, as the ATP-binding pockets of Trk proteins are highly similar.

Compounds 2a22, 2a25, and 4a22 do not Affect the Viability of Cortical Neurons or MG87/TrkB/Luc/Elk1 Cells and are Nontoxic to Rodent Animal Models

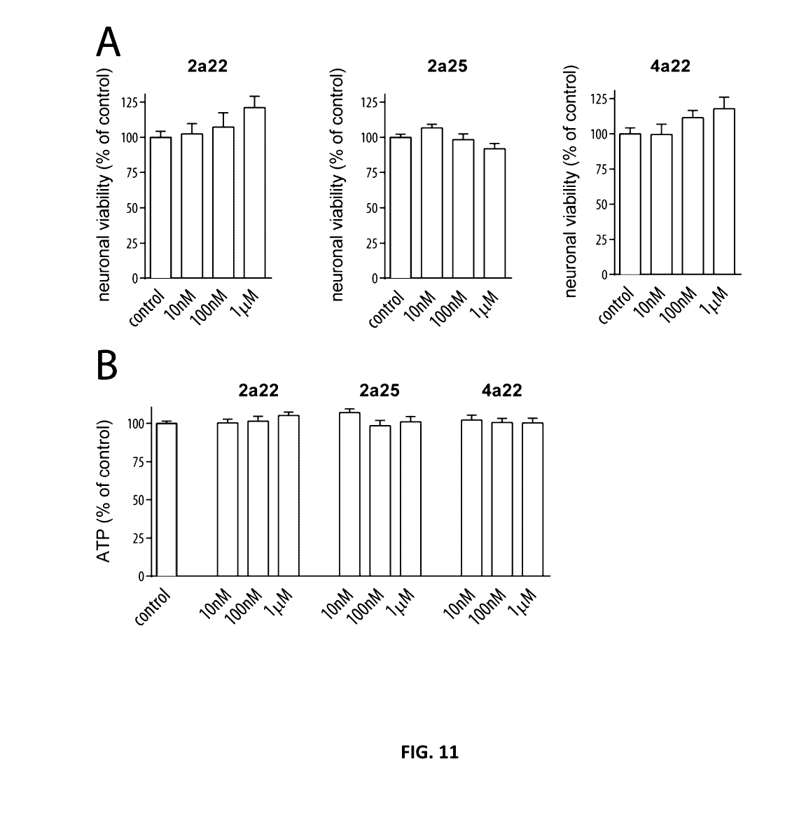

Activation of Trk kinases by neurotrophins has been implicated to be involved in the survival of neuronal cells. For this reason, we tested if Trk inhibitors 2a22, 2a25, and 4a22 can have effect on attenuate the viability of neurons. Rat cortical cells were treated with these compounds at different concentrations for 24 h. No reduction in the number of viable neurons even at 1 μM concentration was observed (FIG. 11, panel A)).

To test the effect of these compounds on the viability of rapidly dividing cells, 2a22, 2a25, and 4a22 were applied at different concentrations to the growth medium of TrkB-expressing MG87/TrkB/luc/Elk1 cells for 24 h, after which the cellular ATP content was quantified. Similarly to the experiment with cortical neurons, no effect of these compounds on cell viability was seen (FIG. 11, panel B).

We also carried out in vivo toxicology studies with the compounds 2a22, 2a25, and 4a22 in C57/B16 mice. These in vivo studies showed that compounds 2a22, 2a25, and 4a22 have no toxic effects in mice.

### Kinase Profiling of 6 Potent Compounds

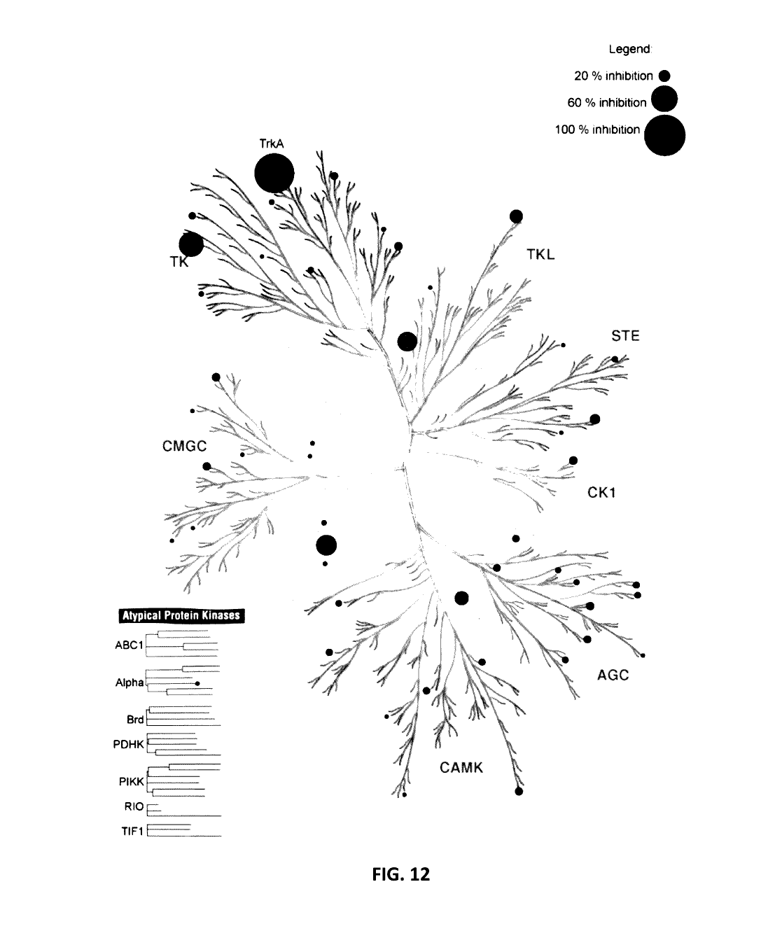

A selection of 48 kinases representing the human kinome (FIG. 12) was used for in vitro kinase profiling to determine the activity of 6 potent TrkA inhibitors that were selected based on the PC-12/luc/Elk1 luciferase assay results (IC₅₀'s around 100 nM or lower). The compounds used were 2a21, 2a22, 2a23, 2a25, 3a25, and 4a22 at 100 nM and 1 μM concentrations (FIGS. 13A-13B). According to the results, at concentration of 100 nM all compounds were relatively TrkA specific inhibiting only up to 3 off-target kinases, while reducing the activity of TrkA by at least 82% at 100 nM concentration and 94% at 1 μM concentration. At 100 nM, 2a25 was able to inhibit only TrkA. 2a23, 3a25, and 4a22 were reducing the activity at least by 50% of only one kinase in addition to the Trk kinases at 100 nM. Kinases that were inhibited by most of the compounds at 1 concentration included Aurora-B, CAMKK2, CHK2, IRAK4, LCK, and MAP3K11.

As compounds 2a21, 2a22, 4a22, and 2a23 have been described as Syk inhibitors, it was initially a surprise that these compounds failed to inhibit Syk at significant levels in our experiments. However, a recent extensive analysis of kinase inhibitors, which also addressed some Syk inhibitors, including the compound named here as 2a21, concluded that three of the tested “Syk inhibitors” had extremely different selectivity among a wide range of kinases and only one of these three compounds was a potent Syk inhibitor. It is noteworthy that our results highly correlate with the results of this study—at 1 μM concentration of 2a21 Gao et al. reported 38% residual activity of Syk and 1% residual activity of TrkA, compared to our results of 47% and 2%, respectively. Thus, 2a21 together with some other compounds described here by us (especially 2a22 and 4a22) are potent and selective Trk inhibitors with no significant effect on Syk.

### Molecular Docking

A conformational search of 4a22 for Z isomers was carried out with MacroModel of Maestro version 9.3, using MMFFs force field in water solution. Geometry optimizations of the obtained conformers in the gas phase were performed with Gaussian 09 program package, using CAM B3LYP functional and 6-31+G* basis set. Frequency analysis was used to confirm whether the structure is a minimum (NImag=0).

The crystal structure of TrkA was downloaded from Protein Data Bank (ID: 4AOJ) with resolution 2.75 Å measured by X-ray diffraction. The protein consisted of a homotrimer of Chain A, Chain B, and Chain C, thus, only Chain A was used. Water molecules were not removed.

AutoDock 4.2.1 was used for the docking studies. All hydrogens were added to the protein. The potential binding partner groups for 4a22 to the TrkA receptor were taken from a previous study and are shown in TABLE 2. The calculated grid maps had dimensions of 41×41×41 points with a spacing of 0.375 Å. Number of Genetic Algorithm was set to 50 runs, other docking parameters were default settings. Genetic Algorithm with Local Search, i.e. Lamarckian GA was used as the docking algorithm.

TABLE 2
Studied binding partner groups in the TrkA receptor.

| Binding site | | | |
|---|---|---|---|
| in TrkA | x-coordinate | y-coordinate | z-coordinate |
| Phe589 O | 89.268 | 60.592 | 29.925 |
| Glu590 O | 91.100 | 57.741 | 29.914 |
| Met592 NH | 94.271 | 56.489 | 30.879 |
| Met592 CB | 94.621 | 54.173 | 30.106 |
| Met592 O | 96.815 | 55.675 | 29.636 |
| Asp596 NH | 98.905 | 52.333 | 24.858 |
| Asp596 O | 100.215 | 49.964 | 24.769 |
| Arg599 O | 105.214 | 48.998 | 24.434 |
| Gly667 NH | 90.110 | 50.125 | 24.146 |

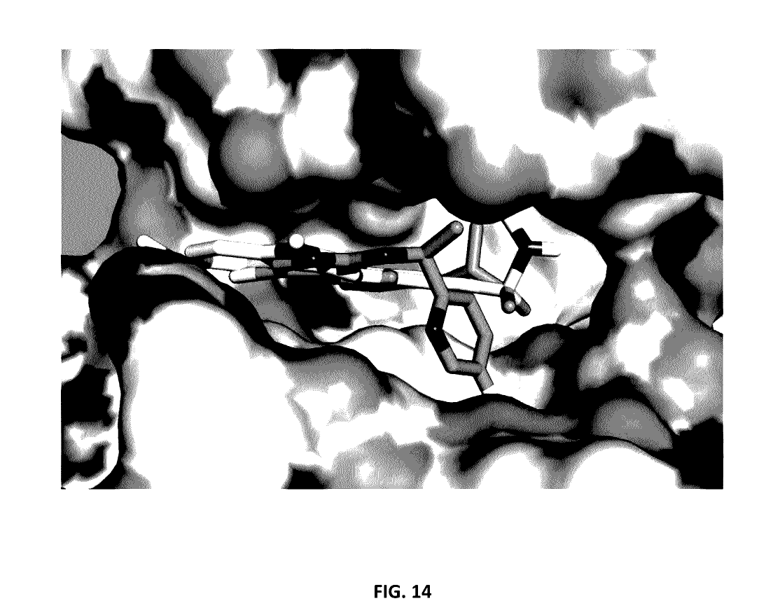

All obtained conformers of 4a22 were used in the docking procedure. The most preferable binding partner was carbonyl oxygen of Met592 for Z isomer. The best corresponding docking score (i.e. AutoDock estimated binding energy) was −9.59 kcal/mol (estimated inhibition constant Kᵢ=92.84 nM) in case of not the lowest-energy conformer, embedded in the pocket (FIG. 14) used in the previous study with AZ-23.

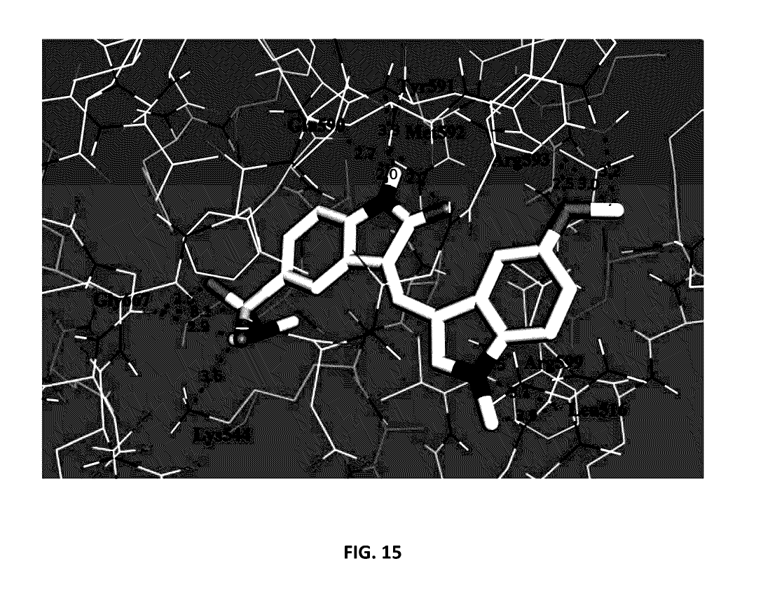

Compound 4a22 binds through the oxindole motif, forming hydrogen bonds to the backbone atoms of residues Glu590 (carbonyl oxygen), Tyr591 (amide NH), and Met592 (amide NH). The sulfonamide group has electrostatic interactions with zwitterion of Lys544 and carbonyl oxygen of Gly667. Indole moiety interacts with the carbonyl oxygen of Leu516 and amine group (NH2) of Arg599. Besides, the hydrogens in methoxy substituent form additional hydrogen bonds with the backbone carbonyl oxygen of Leu516, hydroxyl group of Tyr591, and carbonyl oxygen of Arg593. All corresponding hydrogen-bond (HB) lengths and electrostatic interactions are given in TABLE 3 and shown in FIG. 15.

TABLE 3
Proposed binding partners for 4a22
(Z) in the TrkA ATP-binding site.

| | | Type of interaction |
|---|---|---|
| Length, | | Error! Reference |
| Å | Interaction between atoms | source not found. |
| 2.7 | oxindole, NH . . . O, Glu590 | moderate HB |
| 3.3 | oxindole, NH . . . NH, Tyr591 | weak HB |
| 2.0 | oxindole, HN . . . HN, Met592 | strong HB |
| 2.7 | oxindole, O . . . HN, Met592 | moderate HB |
| 3.6 | sulfonamide, NH . . . NH₃⁺, Lys544 | weak HB |
| 2.5 | sulfonamide, O . . . O, Gly667 | mostly electrostatic |
| 2.9 | sulfonamide, O . . . O, Gly667 | mostly electrostatic |
| 3.1 | sulfonamide, S . . . O, Gly667 | mostly electrostatic |
| 3.1 | indole, H₃C—N . . . O, Leu516 | mostly electrostatic |
| 3.5 | indole, H₃C—N . . . H₂N, Arg599 | weak HB |
| 2.9 | indole, N—CH₃ . . . O, Leu516 | moderate HB |
| 3.2 | indole, O—CH₃ . . . OH, Tyr591 | moderate HB |
| 3.0 | indole, O—CH₃ . . . O, Arg593 | moderate HB |
| 2.5 | indole, H₃C—O . . . O, Arg593 | mostly electrostatic |

### EXAMPLES

### Example I

### Data Set and Methodology

The data on known indole-like TrkA inhibitors were collected from ChEMBL database, using keywords “Nerve growth factor receptor TrkA, Homo sapiens, Homologous protein/Protein, Assay Type B (i.e. biochemical assays)”. The data set consisted of 11 oxindoles and aza-oxindoles, 24 3,5-disubstituted 7-azaindoles, and 14 oxindole amides and ureas (FIG. 1) but one oxindole and one 7-azaindole were discarded because of too high IC₅₀ values (4700 and 3167 nM, respectively). The IC₅₀'s of other compounds were in the range 1.67 to 160 nM. In the further treatment, the IC₅₀ values were transformed into log IC₅₀ units.

The two-dimensional molecular structures of the aforementioned compounds were converted into the three-dimensional structures and preoptimized by built-in minimizer using Maestro 9.3. Conformational search was carried out by the CMol3D program of QSARModel (version 5.0) for the known indole-like compounds, where random conformations were constructed by means of Stochastic Proximity Embedding algorithm followed by optimization based on MMFF94s force field to improve their quality. Thereafter, all geometries were optimized as random vacuum conformer with the minimum potential energy using MOPAC 6.0. The quantum-mechanical semiempirical calculation in the form of the AM1 energy minimization was subsequently applied, using the Polak-Ribiere Conjugate Gradient (PRCG) optimization method with a gradient 0.01 kcal/Å as a stop criterion. The following keywords were used for the optimization procedure: AM1 VECTORS BONDS PI POLAR PRECISE ENPART EF MMOK NOINTER GRAPH GNORM=0.05 XYZ.

The essence of the FQSARModel program is an efficient and rapid generation of totally new compounds from a training set (compounds are fragmentized into linearly connected structural fragments) and automatic prediction of a studied property. In case of the series of compound 2, FQSARModel (version 1.0) was used for the prediction of IC₅₀ values. As conformational search was not carried out by CMol3D program to keep the corresponding isomers, a somewhat better correlation was obtained when the three-dimensional structures of compound 2 and its derivatives were preoptimized by molecular mechanics MM+ field using HyperChem 8.0. Methodology of the geometry optimization in FQSARModel is the same as in the QSARModel. The final step in the FQSAR algorithm is the calculation of descriptors to obtain a descriptor-compounds matrix. The corresponding descriptors were used in the QSAR BMLR model (Equation (3) in TABLE 1) to calculate the IC₅₀ values for the series of compound 2.

### Example II

### Generation of Stable Cell Line with a Sensitive Reporter-Gene System to Monitor TrkA Activity

PC-12 (rat adrenal pheochromocytoma cell line) cells were transfected using LIPOD293 DNA In Vitro Transfection Reagent (SignaGen) with 1 μg of pFA2-Elk1 plasmid and 4 μg of pFR-LUC plasmid (PathDetect Elk1 trans-Reporting System; Agilent Technologies). pFA2-Elk1 plasmid codes for the fusion protein consisting of GAL4-dbd (DNA-binding domain) followed by Elk1 transcription factor and contains Geneticin G418 resistance gene neomycin. pFR-LUC plasmid codes for GAL4 upstream activation sequence (UAS) followed by luciferase reporter gene. A puromycin resistance gene was introduced to the pFR-LUC plasmid using Bst1107I and NdeI restrictases from pGL4.22[luc2CP/Puro] (Promega). The selection of transfected cells was initiated two days after the transfection with addition of 300 μg/ml of G418 (Sigma) and 0.75 μg/ml of puromycin (Sigma-Aldrich) and continued for about one and a half months until distinct cell colonies could be picked, plated, and tested for responsiveness to NGF. Generated cell line, called hereafter PC-12/luc/Elk1, was maintained in Dulbecco's Modified Eagle's Medium (DMEM; PAA) containing 6% fetal bovine serum (FBS; PAA), 6% horse serum (HS; PAA), 1% penicillin/streptomycin (PS; Gibco), 300 μg/ml of Geneticin G418, and 0.75 μg/ml of puromycin. MG87/TrkB/luc/Elk1 and MG87/par/luc/Elk1Error! Reference source not found. were maintained in Minimum Essential Media (MEM; PAA) 10% FBS, 1% PS, 2 μg/ml Blasticidin (PAA), and 500 μg/ml G418. PC-12 cells were maintained in DMEM containing 6% FBS, 6% HS, and 1% PS.

### Example III

### Determination of the Z′-Factor of the PC-12/Luc/Elk1 Cell-Line

PC-12/luc/Elk1 cells were plated one day before the assay on 96 well plates in 25,000 cells per well. Next day the growth media was changed to 50 ng/ml of NGF (Peprotech) together with 0.1% dimethyl sulfoxide (DMSO; Sigma-Aldrich; negative control) or to 50 ng/ml of NGF together with 5 nM of a known TrkA inhibitor AZ-23Error! Reference source not found. (Axon Medchem; positive control) in 100 μl of DMEM 6% HS; 6% FBS; 1% PS. Three time points were chosen to be tested (24 h, 18 h, and 6 h) with 16 wells per effector per time point. After 24 h, 18 h or 6 h the growth media was removed and 20 μl of Steady Glo Assay Reagent (Promega) was added to each well. The plate was subjected to 10 minutes of shaking and, thereafter, analyzed using TECAN plate reader. The results were used to calculate the Z′-factor with the Equation (4)

$${Z}^{′} = 1 - \frac{( 3 {σ}_{+} + 3 {σ}_{-} )}{\vert {μ}_{+} - {μ}_{-} \vert} , \qquad ( Equation 4 )$$

where σ₊ and σ₋ are the standard deviations of the positive and negative control, and μ₊ and μ₋ their means.

### Example IV

### Determination of IC₅₀ of Compounds in Cellular Context

Based on virtual screening for new scaffolds, one potential compound (Z)-3-((5-methoxy-1H-indol-3-yl)methylene)-2-oxindole (2) was selected for the further research. Several derivatives of this compound were ordered (FIGS. 2A-2G). The IC₅₀'s of the compounds were determined using cellular and biochemical assays. In the cellular assays, using PC-12/luc/Elk1 and MG87/TrkB/luc/Elk1 cells, the compounds were assessed in gradual dilutions of the compounds in triplets together with NGF or BDNF (Peprotech), respectively. TrkA inhibition was determined using same methodology as for measuring the Z′-factor with 18 h treatment time. The MG87/TrkB/luc/Elk1 cells were plated one day prior to the experiment, 15,000 cells per well on a 96-well plate. BDNF (50 ng/ml) together with 0.1% DMSO was used as the negative control and BDNF with 5 nM of AZ-23 (28) as the positive control in 100 μl of MEM 10% FBS, 1% PS. Thereafter, the assay was conducted likewise as with PC-12/luc/Elk1 cells.

Based on cellular assays, the statistical calculations for determining IC₅₀ values were performed using R statistical programming software. Percent inhibition values for each compound together with log transformed drug concentrations were fitted to 4 parameter non-linear logistic model, which was used to calculate IC₅₀ values for each compound. The formula used for calculation of percent inhibition for treatments was

$$\frac{{X}_{max} - {X}_{i}}{{X}_{max}} , \qquad ( Equation 5 )$$

where Xᵢ—luciferase signal in the presence of compound together with NGF or BDNF (inhibitory activity), Xₘₐₓ—luciferase signal in DMSO 0.1% and NGF or BDNF treated cells (normal activity).

The biochemical kinase assays were custom ordered from ProQinase, Germany.

### Example V

### Western Blotting

The PC-12 and MG87/TrkB/luc/Elk1 cells were plated on 6-well plates one day prior to the assay. Cells were treated for 10 minutes with compounds and simultaneously with 50 ng/ml of NGF (PC-12 cells) or BDNF (MG87/TrkB/luc/Elk1 cells). 0.1% of DMSO was used to determine the base level, 0.1% of DMSO concurrently with 50 ng/ml of NGF or BDNF served as a negative control and 0.1% of DMSO concurrently with 5 nM of AZ-23 as a positive control. Thereafter, western blotting was performed as described previously. Antibodies used included: rabbit anti-TrkA (#06-574; 1:1000) from Millipore; rabbit anti phospho-TrkA (#9141; 1:1000), rabbit anti phospho-TrkA/TrkB (#4619; 1:1000), rabbit anti-TrkB (#4603; 1:1000) and rabbit anti phospho-Akt (#4058; 1:3000) from Cell Signaling; mouse anti phosphor-Erk1/2 (#sc-7383; 1:1000) from Santa Cruz and anti-tubulinβ clone E7 (1:1000) from Prof. Michael Klymkovsky.

### Example VI

### Cell Viability Assessment

Primary cultures of rat cortical cells were prepared from neonatal Wistar rats as previously described by P. Wareski, J. Biol. Chem, 284 (2009) 21379-21385. Neurons were grown in NEUROBASAL, a medium supplemented with B27 with phenol red on ploy-L-lysine-coated 35 mm glass bottomed dishes. Culture media and supplements were obtained from Invitrogen (Carlsbad, Calif.). For viability measurements neurons were first transfected with neuronal maker, pAAV-hSyn-DsRedExpress obtained from Addgene (Cambridge, Mass.) allowing better to assess the morphology of individual neurons. Briefly, the condition medium was replaced with 100 μL Opti-MEM 1 medium, containing 2% Lipofectamine 2000 and 1-2 μg of total DNA. Neurons were incubated for 3-4 hrs, after which fresh medium was added. 3 days later the neurons treated with compounds 2a23, 2a25, and 4a22 at different concentrations for 24 hr, after which the neuronal number was counted from 8 to 12 dishes per treatment group (at least 50 randomly chosen fields from each dish).

For measurement of ATP content, the MG87/TrkB/luc/Elk1 cells were grown on 96-well white plates, treated with 2a22, 2a25, and 4a22 at different concentrations for 24 hr and the cellular ATP content as quantified using CELLTITER-GO Luminescent Cell Viability Assay (Promega, Sweden) according to manufacturer's recommendations. ATP was measured at least from 8 different wells per treatment condition.

### Example VII

### Synthesis of Compound 2a23

### (Z)-3-((5-methoxy-1H-indol-3-yl)methylene)-2-oxindole-5-sulfonamide

2-oxindole-5-sulfonamide (47 mg, 0.22 mmol) and 5-methoxy-1H-indole-3-carbaldehyde (42 mg, 0.24 mmol) were suspended in absolute ethanol (0.44 mL) and piperidine (6.5 μl, 0.066 mmol) was added. Mixture was heated to 70° C. for 2 hours. Additional amount of ethanol (0.44 mL) was added and mixture heated for further 3 hours. Reaction was allowed to cool to ambient temperature and product that precipitated was filtered out as yellow-brown solid (74 mg, 91%) (d.r=7.5:1 (Z/E)). ¹H NMR (400 MHz, DMSO-d₆) δ 12.03 (s, 1H), 10.90 (s, 1H), 9.48 (s, 1H), 8.35 (d, J=1.8 Hz, 1H), 8.28 (s, 1H), 7.75 (d, J=2.4 Hz, 1H), 7.63 (dd, J=8.2, 1.8 Hz, 1H), 7.42 (d, J=8.7 Hz, 1H), 7.15 (s, 2H), 6.97 (d, J=8.1 Hz, 1H), 6.88 (dd, J=8.7, 2.4 Hz, 1H), 3.89 (s, 3H).

### Example VIII

### Synthesis of Compound 3a23

### (Z)-3-((5-hydroxy-1H-indol-3-yl)methylene)-2-oxindole-5-sulfonamide

2-oxindole-5-sulfonamide (47 mg, 0.22 mmol) and 5-hydroxy-1H-indole-3-carbaldehyde (39 mg, 0.24 mmol) were suspended in absolute ethanol (0.88 mL) and piperidine (6.5 μl, 0.066 mmol) was added. Mixture was heated to 70° C. for 3 hours, then allowed to cool to ambient temperature. Product precipitated as brown solid and was filtered out and dried in vacuum (65 mg, 83%) (d.r=6:1 (Z/E)). ¹H NMR (400 MHz, DMSO-d₆) δ 11.95 (s, 1H), 10.87 (s, 1H), 9.38 (s, 1H), 9.04 (s, 1H), 8.26 (d, J=1.8 Hz, 1H), 8.11 (s, 1H), 7.60 (dd, J=8.1, 1.8 Hz, 1H), 7.46 (d, J=2.2 Hz, 1H), 7.33 (d, J=8.5 Hz, 1H), 7.13 (s, 2H), 6.96 (d, J=8.2 Hz, 1H), 6.77 (dd, J=8.6, 2.3 Hz, 1H).

### Example IX

### Synthesis of Compound 2a25

### (Z)-3-((5-methoxy-1-methyl-1H-indol-3-yl)methylene)-N,N-dimethyl-2-oxindole-5-sulfonamide

N,N-dimethyl-2-oxindole-5-sulfonamide (48 mg, 0.20 mmol) and 5-methoxy-1-methyl-1H-indole-3-carbaldehyde (42 mg, 0.22 mmol) were suspended in absolute ethanol (0.8 mL) and piperidine (6 μl, 0.060 mmol) was added. Mixture was heated to 70° C. for 4 hours and then cooled to ambient temperature. Product precipitated and was filtered out and dried in vacuum to yield 71 mg of product (86%) as yellow solid (d.r=20:1 (Z/E)). ¹H NMR (400 MHz, DMSO-d₆) δ 11.01 (s, 1H), 9.49 (s, 1H), 8.39 (s, 1H), 8.32 (d, J=1.8 Hz, 1H), 7.90 (d, J=2.4 Hz, 1H), 7.60-7.46 (m, 2H), 7.05 (d, J=8.2 Hz, 1H), 6.98 (dd, J=8.8, 2.4 Hz, 1H), 3.94 (s, 3H), 3.91 (s, 3H), 2.65 (s, 6H).

### Example X

### Synthesis of Compound 3a25

### (Z)-3-((5-hydroxy-1-methyl-1H-indol-3-yl)methylene)-N,N-dimethyl-2-oxindole-5-sulfonamide

N,N-dimethyl-2-oxindole-5-sulfonamide (48 mg, 0.20 mmol) and 5-hydroxy-1-methyl-1H-indole-3-carbaldehyde (39 mg, 0.22 mmol) were suspended in absolute ethanol (0.8 mL) and piperidine (6 μl, 0.06 mmol) was added. Mixture was heated to 70° C. for 4 hours and then cooled to ambient temperature. Product precipitated and was filtered out and dried in vacuum to yield 51 mg of product (64%) as yellow solid (d.r=20:1 (Z/E)). ¹H NMR (400 MHz, DMSO-d₆) δ 10.96 (s, 1H), 9.38 (s, 1H), 9.10 (s, 1H), 8.36-8.24 (m, 2H), 7.67 (d, J=2.3 Hz, 1H), 7.50 (dd, J=8.2, 1.8 Hz, 1H), 7.38 (d, J=8.8 Hz, 1H), 7.02 (d, J=8.1 Hz, 1H), 6.83 (dd, J=8.7, 2.3 Hz, 1H), 3.89 (s, 3H), 2.63 (s, 6H).

### Example XI

### Synthesis of Compound 4a22

### (Z)-3-((5-methoxy-1-methyl-1H-indol-3-yl)methylene)-N-methyl-2-oxindole-5-sulfonamide

N-methyl-2-oxindole-5-sulfonamide (36 mg, 0.16 mmol) and 5-methoxy-1-methyl-1H-indole-3-carbaldehyde (33 mg, 0.18 mmol) were suspended in absolute ethanol (0.64 mL) and piperidine (4.8 μl, 0.05 mmol) was added. Mixture was heated to 70° C. for 4 hours and then cooled to ambient temperature. Product precipitated and was filtered out and dried in vacuum to yield 47 mg of product (74%) as yellow solid (d.r. 20:1 (Z/E)). ¹H NMR (400 MHz, DMSO-d₆) δ 10.95 (s, 1H), 9.45 (s, 1H), 8.31 (d, J=2.9 Hz, 2H), 7.83 (d, J=2.4 Hz, 1H), 7.56 (dd, J=8.2, 1.8 Hz, 1H), 7.50 (d, J=8.8 Hz, 1H), 7.17 (q, J=5.1 Hz, 1H), 7.00 (d, J=8.1 Hz, 1H), 6.95 (dd, J=8.9, 2.4 Hz, 1H), 3.92 (s, 3H), 3.90 (s, 3H), 2.44 (d, J=5.0 Hz, 3H).

What is claimed is:

1. A composition comprising formula (I) or a pharmaceutically acceptable salt thereof 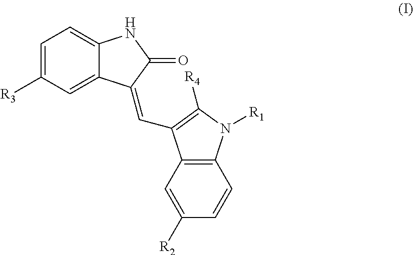
wherein:
R1 is a lower alkyl having 1 to 6 carbons;
R2 is a hydroxy or lower alkoxy having 1 to 6 carbons;
R3 is SO₂N(CH₃)₂; and
R4 is H.

2. The composition of claim 1, wherein R1 is CH₃ and R2 is OCH₃.

3. The composition according to claim 1, wherein R1 is CH₃ and R2 is OH.

4. A method of inhibiting kinase activity of tropomyosin receptor kinase (Trk), the method comprising providing a cell population expressing a Trk, adding the composition according to claim 1 to the cell population, and optionally exposing the cell population to an agonist of the Trk, further wherein R1 is CH₃ and R2 is OCH₃ or OH.

5. The method of claim 4, wherein the Trk is selected from the group consisting of TrkA, TrkB and TrkC, and wherein the composition is added in an amount sufficient to inhibit kinase activity of the Trk to 18% or less residual activity, optionally to 10% or less residual activity.

6. A method of inhibiting kinase activity of a tropomyosin receptor kinase (Trk), the method comprising providing a cell population expressing a Trk, and adding to the cell population a composition comprising the Z isomer of formula (I) or the E isomer of formula (II) in an amount sufficient to inhibit kinase activity of the Trk, and optionally exposing the cell population to a Trk agonist, wherein 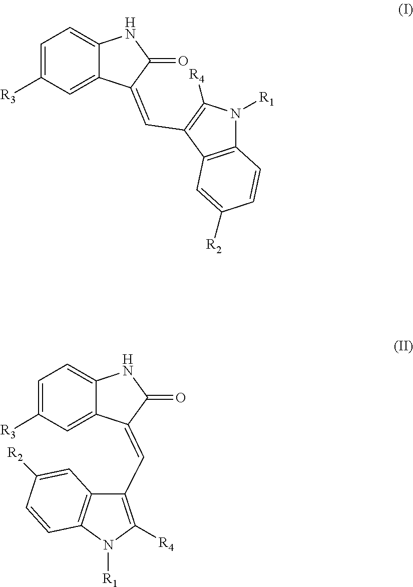
Further wherein:
R1 is CH₃;
R2 is H or OCH₃;
R3 is selected from the group consisting of H, SO₂NH₂ or SO₂NH(CH₃); and
R4 is H.

7. The method of claim 6, wherein the composition is provided in a pharmaceutically acceptable carrier.

8. The method of claim 6, wherein the isomer is the Z isomer, further wherein R3 is SO₂NH₂.

9. The method of claim 8, wherein the Trk is selected from the group consisting of TrkA, TrkB and TrkC and, wherein the composition is added in an amount sufficient to inhibit kinase activity of the Trk to 10% or less residual activity, optionally to 3% or less residual activity.

10. The method of claim 6, wherein the isomer is the Z isomer, further wherein R1 is H, R2 is OCH₃, and R3 is SO₂NH₂.

11. The method of claim 10, wherein the Trk is selected from the group consisting of TrkA, TrkB, and TrkC, wherein and the composition is added in an amount sufficient to inhibit kinase activity of the Trk to 10% or less residual activity, optionally to 6% or less residual activity.

12. The method of claim 6, wherein the isomer is the Z isomer, further wherein R1 is CH₃, R2 is OCH₃, and R3 is SO₂NH(CH₃).

13. The method of claim 12, wherein the Trk is selected from the group consisting of TrkA, TrkB, and TrkC, and wherein the composition is added in an amount sufficient to inhibit kinase activity of the Trk to 10% or less residual activity, optionally to 4% or less residual activity.

14. A method of inhibiting kinase activity in a cell, the method comprising providing a cell population expressing at least one kinase selected from the group consisting of CaMKK2, Map3K11, TrkA, TrkB, and TrkC; and adding to the cell population the composition of claim 2 in an amount sufficient to inhibit kinase activity; and optionally exposing the cell population to an agonist of the kinase.

15. A method of inhibiting kinase activity in a cell, the method comprising providing a cell population expressing at least one functional kinase selected from the group consisting of Aurora-B, CaMKK2, Lck, TrkA, TrkB, and TrkC; and adding to the cell population the composition of claim 3 in an amount sufficient to inhibit kinase activity; and optionally exposing the cell population to an agonist of the kinase.

16. A method of inhibiting kinase activity in a cell, the method comprising providing a cell population expressing at least one functional kinase selected from the group consisting of Aurora-B, CaMKK2, Chk2, Erb-B4, Irak4, Lck, Map3K11, Ripk2, Sgk1, Syn aa1-635; and adding to the cell population a composition comprising a compound of the formula (I), optionally in a pharmaceutically acceptable carrier, in an amount sufficient to inhibit kinase activity; and optionally exposing the cell population to an agonist of the kinase, wherein formula (I) is 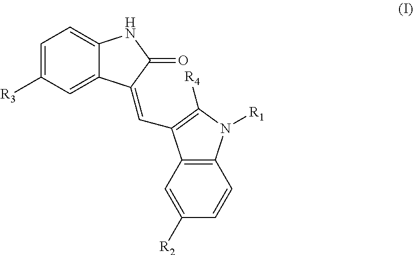
further wherein:
a) if the kinase is Aurora-B,
i) R1 is CH₃, R2 is H, and R3 is SO₂NH₂, and R4 is H; or
ii) R1 is CH₃, R2 is OCH₃, and R3 is SO₂NH₂, and R4 is H; or
iii) R1 is H, R2 is OCH₃, R3 is SO₂NH₂, and R4 is H;
b) if the kinase is CAMKK2,
i) R1 is CH3, R2 is H, and R3 is SO₂NH₂, and R4 is H; or
ii) R1 is CH₃, R2 is OCH₃, and R3 is SO₂NH₂, and R4 is H; or
iii) R1 is H, R2 is OCH₃, R3 is SO₂NH₂, and R4 is H; or
iv) R1 is CH3, R2 is OCH₃, and R3 is SO₂NH(CH₃), and R4 is H;
c) if the kinase is CHK2,
i) R1 is CH3, R2 is H, and R3 is SO₂NH₂, and R4 is H; or
ii) R1 is CH₃, R2 is OCH₃, and R3 is SO₂NH₂, and R4 is H; or
iii) R1 is H, R2 is OCH₃, R3 is SO₂NH₂, and R4 is H;
d) if the kinase is ERBB4, R1 is CH3, R2 is OCH₃, R3 is SO₂NH(CH₃), and R4 is H;
e) if the kinase is IRAK4,
i) R1 is CH3, R2 is H, and R3 is SO₂NH₂, and R4 is H; or
ii) R1 is CH₃, R2 is OCH₃, and R3 is SO₂NH₂, and R4 is H; or
iii) R1 is H, R2 is OCH₃, R3 is SO₂NH₂, and R4 is H; or
iv) R1 is CH3, R2 is OCH₃, and R3 is SO₂NH(CH₃), and R4 is H;
f) if the kinase is LCK,
i) R1 is CH3, R2 is H, and R3 is SO₂NH₂, and R4 is H;
ii) R1 is CH3, R2 is OCH₃, and R3 is SO₂NH(CH₃), and R4 is H;
g) if the kinase is MAP3K11,
i) R1 is CH3, R2 is H, and R3 is SO₂NH₂, and R4 is H; or
ii) R1 is CH₃, R2 is OCH₃, and R3 is SO₂NH₂, and R4 is H; or
iii) R1 is H, R2 is OCH₃, R3 is SO₂NH₂, and R4 is H; or
h) if the kinase is RIPK2,
i) R1 is H, R2 is OCH₃, R3 is SO₂NH₂, and R4 is H; or
i) if the kinase is SGK1,
i) R1 is H, R2 is OCH₃, R3 is SO₂NH₂, and R4 is H; or
j) if the kinase is SYN aa1-635,
i) R1 is H, R2 is OCH₃, R3 is SO₂NH₂, and R4 is H.

17. An engineered cell line for monitoring modulation of a tropomyosin receptor kinase (Trk), the cell line is a transgenic population of rat adrenal pheochromocytoma (PC12) cells, wherein the PC12 cells comprise:
a) a tropomyosin receptor kinase selected from the group consisting of TrkA, TrkB and TrkC and
b) a first plasmid comprising a first promoter and encoding a GALA-DNA binding domain operatively connected to Elk1 transcription factor, the first plasmid further comprising a first antibiotic resistance gene in operative alignment with the promoter; and
c) a second plasmid comprising, in operative alignment, a second promoter, a GAL4 upstream activation sequence (UAS), a luciferase reporter gene, and a second antibiotic resistance gene.

18. A method of screening for inhibition of Trk in an engineered cell line, the method comprising:
a) culturing the engineered cell line of claim 17 in cell culture medium;
b) exposing the cell line to an agonist of Trk;
c) adding an inhibitor of Trk to the cell line; and
d) monitoring the cell line for luciferase expression.

19. The method of claim 18, wherein the engineered cell line is cultured in a plurality of cell cultures and the inhibitor is added at different concentrations to different cell cultures, the method further comprising determining an IC50 of the inhibitor.
